(19) United States
(12) Patent Application Publication
(10) Pub. No.: US 2024/0117010 A1
(43) Pub. Date: Apr. 11, 2024

(54) BINDING PROTEIN SPECIFIC FOR THE SPIKE PROTEIN OF SEVERE ACUTE RESPIRATORY SYNDROME CORONA VIRUS 2 (SARS-COV-2)

(71) Applicant: Navigo Proteins GmbH, Halle/Saale (DE)

(72) Inventors: Mathias Kahl, Halle/Saale (DE); Erik Fiedler, Halle/Saale (DE); Eva Bosse-Doenecke, Halle/Saale (DE); Anja Katzschmann, Halle/Saale (DE); Hanna Bobolowski, Halle/Saale (DE); Jonathan Lotze, Halle/Saale (DE); Maren Meysing, Halle/Saale (DE)

(73) Assignee: Navigo Proteins GmbH, Halle/Saale (DE)

(21) Appl. No.: 18/012,284

(22) PCT Filed: Jun. 23, 2021
(86) PCT No.: PCT/EP2021/067225

§ 371 Date: Dec. 22, 2022

(30) Foreign Application Priority Data
Jun. 23, 2020 (EP) 20181725.1
Jul. 17, 2020 (EP) 20186603.5
Jul. 31, 2020 (EP) 20188821.1
Oct. 12, 2020 (EP) 20201357.9

(51) Int. Cl. C07K 16/10; G01N 33/569

(52) U.S. Cl. CPC C07K 16/1003; G01N 33/56983; C07K 2317/92

(57) ABSTRACT

The present invention relates to novel proteins that specifically bind to the spike protein or domains thereof of the severe acute respiratory syndrome corona virus 2 (SARS-Cov-2) or variants of SARS-Cov-2. The proteins of the present invention represent advanced and powerful tools, for example for the purification of the virus or a vaccine for the virus, by virtue of said binding affinity for spike protein or domains of the spike protein of SARS-Cov-2 or variants thereof. Thus, the novel proteins of the present invention are particularly advantageous because they allow precise capturing of proteins or particles comprising spike proteins, S1 domain, and/or RBD in affinity chromatography. Further, the novel proteins of the present invention can be used in medical applications caused by or related to SARS-Cov-2 or variants thereof.

### CROSS REFERENCE TO RELATED APPLICATIONS

The present application is a United States National Stage Application of PCT International Patent Application Serial No. PCT/EP2021/067225, filed Jun. 23, 2021, which itself claims the benefit of the following European Patent Applications: EP 20181725.1, filed Jun. 23, 2020; EP 20186603.5, filed Jul. 17, 2020; EP 20188821.1, filed Jul. 31, 2020; and EP 2021357.9, filed Oct. 12, 2020. The disclosure of each of these applications is incorporated herein by reference in its entirety.

### TECHNICAL FIELD

The present invention relates to novel proteins that specifically bind to the spike protein or domains thereof of the severe acute respiratory syndrome corona virus 2 (SARS-Cov-2) or variants of SARS-Cov-2. The proteins of the present invention represent advanced and powerful tools, for example for the purification of the virus or a vaccine for the virus, by virtue of said binding affinity for spike protein or domains of the spike protein of SARS-Cov-2 or variants thereof. Thus, the novel proteins of the present invention are particularly advantageous because they allow precise capturing of proteins or particles comprising spike proteins, S1 domain, and/or RBD in affinity chromatography. Further, the novel proteins of the present invention can be used in medical applications caused by or related to SARS-Cov-2 or variants thereof.

### BACKGROUND OF THE INVENTION

The severe acute respiratory syndrome corona virus 2 (SARS-Cov-2) is the cause for the pandemic virus disease COVID-19. The SARS-Cov-2 virus binds via glycoprotein S (spike protein) to the angiotensin-converting enzyme ACE2 receptor of the host cell. Functionally, the spike protein is characterized by two domains: S1 and S2. The S1 domain is responsible for the binding of the ACE2 receptor on the host cell; the species variable receptor binding domain (RBD) is located within the S1 domain. After binding of S1 domain to ACE2, the spike protein in cleaved in S1 domain and S2 domain by a serine protease TMPRSS2. The S2 domain is responsible for the fusion of the virus to the cell membrane which results in entry to the host cell. Upon entry, the viral genome is released to the cytoplasm of the host cell and can be translated directly from the cellular translation complex. New virus particles are assembled and exported as cause for the pandemic disease COVID-19 with severe and less-severe symptoms.

To stop the pandemic, at least 2/3 of the population should be immune. Therefore, the development of a vaccination against the virus is currently of great interest. The spike protein is the primary target in the development of vaccines, in particular the S1 domain or receptor binding domain (RBD) of the spike protein. Therefore, it will be of great importance to have efficient and reliable methods for the purification of spike protein (or domain thereof) based vaccines. Further, early detection of the virus or variants of the virus is of highest interest, as well as novel therapeutic options.

The present invention meets this need by providing novel binding proteins for the spike protein of SARS-Cov-2. These novel binding proteins are particularly advantageous because they allow a precise capturing in affinity chromatography as well as potential applications in medicine, particularly in diagnostic applications or in therapeutic applications. The above overview does not necessarily describe all problems solved by the present invention.

### SUMMARY OF THE INVENTION

The present disclosure provides the following items 1 to 15, without being specifically limited thereto:
1. A binding protein for the spike protein of severe acute respiratory syndrome corona virus 2 (SARS-Cov-2) comprising an amino acid sequence with at least 80% sequence identity, preferably at least 92% sequence identity, to any one selected from the group of SEQ ID NOs: 2, 4, 5, 6, 23, 40, 41, and 42, wherein the binding protein has a binding affinity of less than 500 nM for the spike protein or domains thereof.
2. The binding protein for the spike protein according to item 1, wherein the binding protein has a binding affinity of less than 500 nM for the S1 domain of the spike protein, preferably wherein the binding protein has a binding affinity of less than 100 nM for the S1 domain of the spike protein. In various embodiments, the binding protein has a binding affinity of less than 500 nM, preferably less than 100 nM, for the receptor binding domain (RBD) of the spike protein.
3. The binding protein for the spike protein according to 1 or 2, wherein 2, 3, 4, 5, or 6 binding proteins for the spike protein are linked to each other.
4. The binding protein for the spike protein according to item 3, wherein the binding protein is a homo-multimer or a hetero-multimer.
5. The binding protein for the spike protein according to item 4, wherein the binding protein is a hetero-multimer, preferably a hetero-dimer, that comprises an amino acid sequence with at least 92% sequence identity, to any one selected from the group of SEQ ID NOs: 2, 40, 41 linked to an amino acid sequence with at least at least 92% sequence identity, to any one selected from the group of SEQ ID NOs: 4, 5, 6, 23, and 42.
6. The binding protein for the spike protein according to item 5, wherein the binding protein comprises amino acid sequences with at least 80%, preferably at least 92%, sequence identity to any one of SEQ ID NOs: 7, 8, 17, 43-47, 66, and 67.
7. The binding protein for the spike protein according to items 1-6, wherein the binding protein is fused to or conjugated to at least one further molecule, preferably selected from the group of (a) non-Immunoglobulin (Ig)-binding protein, (b) a diagnostically active moiety, optionally selected from a radionuclide, fluorescent protein, photosensitizer, dye, or enzyme, or any combination of the above (c) a therapeutically active moiety, optionally selected from a monoclonal antibody or a fragment thereof, a binding protein, a receptor or receptor domain, a receptor binding ligand or antagonist, a radionuclide, a cytotoxic compound, a cytokine, a chemokine, an enzyme, or derivatives thereof, or any combination of the above.
8. The binding protein for the spike protein according to any one of items 1-7 for use in medical applications, preferably for use in the diagnosis or treatment of diseases related to or caused by SARS CoV-2 or variants thereof.
9. The binding protein for the spike protein according to any one of items 1-7 for use in technical applications such as affinity purification of a spike protein or a protein comprising a spike protein domain or a particle containing a spike protein or domain thereof.
10. An affinity separation matrix comprising a binding protein for the spike protein according to any one of items 1-7.
11. Use of the binding protein for the spike protein according to any one of items 1-7, or the affinity separation matrix according to item 9 for affinity purification of a spike protein or a protein comprising a spike protein domain or a particle containing a spike protein or domain.
12. A method of affinity purification of spike protein or a domain thereof or of a particle comprising spike protein or a domain thereof, optionally a virus particle, the method comprising: (a) providing a liquid that contains a spike protein or a domain thereof (e.g. a particle comprising spike protein or domain); (b) providing an affinity separation matrix comprising at least one binding protein for spike protein or a domain thereof according to any one of items 1-6 coupled to said affinity separation matrix; (c) contacting said affinity separation matrix with the liquid under conditions that permit binding of the at least one binding protein for spike protein or a domain thereof according to any one of items 1-6; and (d) eluting said spike protein or a domain thereof or particle comprising the spike protein or domain thereof from said affinity purification matrix.
13. Use of the binding protein for the spike protein according to items 1-7, in methods to determine the presence of the spike protein or a domain thereof, or to determine the presence of a particle comprising the spike protein or domain thereof.
14. A method of analyzing the presence of spike protein or a domain thereof or particle comprising a spike protein or domain thereof in liquid samples, the method comprising the following steps:
   (i) providing a liquid that contains spike protein or a domain thereof or particle comprising a spike protein or domain,
   (ii) providing the binding protein for the spike protein according to items 1-7,
   (iii) contacting the liquid of (i) with the binding protein for the spike protein or a domain thereof according to items 1-7 under conditions that permit binding of the binding protein to the spike protein or a domain thereof,
   (iv) isolating the complex of spike protein or a domain thereof or particle and the binding protein for the spike protein or a domain thereof according to items 1-7, and
   (v) determining the amount of the binding protein for the spike protein according to items 1-7 in the liquid of (i).
15. A polynucleotide encoding the binding protein according to any one of items 1-7. This summary of the invention is not limiting, and other aspects and embodiments of the invention will become evident from the following description, examples and drawings.

### BRIEF DESCRIPTION OF THE FIGURES

FIG. 1 shows the amino acid sequences of binding proteins for the spike glycoprotein (and domains thereof) of SARS-Cov-2. FIG. 1B shows amino acid sequences of dimeric spike protein binding proteins.

FIG. 2 shows a competitive binding study (SPR).

FIG. 2A shows the binding of 212434 (SEQ ID NO: 1) to S1 and to RBD. If S1 and ACE2 or if RBD and ACE2 are applied at the same time to 212434, almost no binding is observed. It is concluded that 212434 binds to the same or an overlapping epitope for S1 or RBD and ACE. FIG. 2B shows the binding of 212361 (SEQ ID NO: 7) to S1 and to RBD. If S1 and ACE2 or if RBD and ACE2 are applied at the same time to 212361, almost no binding is observed. It is concluded that 212361 binds to the same or an overlapping epitope for S1 or RBD and ACE.

FIG. 2C shows the binding of 212335 (SEQ ID NO: 8) to S1 (thick line). If S1 and ACE2 are applied at the same time, no binding is observed due to competitive binding to an overlapping epitope (dotted line).

FIG. 2D shows the binding of 213103 (SEQ ID NO: 9) to 51 (thick line). If 51 and ACE2 are applied at the same time (dotted line), a reduced binding is observed. It is concluded that the spike protein binding protein 213103 binds to a different or only partially overlapping epitope for 51 and ACE.

FIG. 3 shows the detection of SARS-CoV2-S1 binding protein in Protein A ELISA. SARS-CoV2-S1 binding proteins were analyzed for detection in protein A detection ELISA kit to evaluate possible ligand leaching detection. All fusion proteins show comparable detectability to internal rProtein A Kit standard (“rProtein A”).

FIG. 4 shows the elution pH profile of fusion protein 217799 (based on SEQ ID NO: 2 and SEQ ID NO: 5) in AIC experiments. Expressed SARS-CoV2-RBD-His target in Expi293-F cells was applied on packed resin with immobilized 217799 (UV280 nm: solid black line). Elution was performed by decreasing pH (pH trace: solid light grey line) with linear gradient (concentration % B: dashed dark grey line) from pH 6.0 to 2.0. Elution pH was determined by pH of fractions containing the target protein.

FIG. 5 shows caustic stability of selected ligands after 0.1 M NaOH treatment for 10 h.

### DETAILED DESCRIPTION OF THE INVENTION

The present invention provides novel proteins having specific binding affinity for the spike glycoprotein of SARS-Cov-2. The proteins of the present invention represent advanced and powerful tools, for example for the purification of the virus or a protein-based vaccine for the virus, by virtue of said binding affinity for spike protein or domains of the spike protein of SARS-Cov-2 or variants thereof. Thus, the novel proteins of the present invention are particularly advantageous because they allow precise capturing of spike proteins or proteins comprising S1 domain or proteins comprising RBD, or particles containing those viral proteins, in affinity chromatography, and allow precise capturing of vaccines based on spike proteins or proteins comprising S1 domain or proteins comprising RBD, or particles containing those viral proteins, in affinity chromatography. Further, the novel proteins of the present invention can be used in medical applications related to SARS-Cov-2 or variants thereof. The binding affinity for the spike protein or domains thereof is given by a polypeptide comprising SEQ ID NO: 1, SEQ ID NO: 2, SEQ ID NO: 3, SEQ ID NO: 4, SEQ ID NO: 5, or SEQ ID NO: 6, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 37, SEQ ID NO: 40, SEQ ID NO: 41, SEQ ID NO: 42, or an amino acid sequence with at least 80% identity, preferably at least 90%, 91%, 92%, 93%, 94%, or 95%, identity to any one of SEQ ID NOs: 1-6, 20-28, 37, and 40-42. In the present invention, the binding affinity for the spike protein or domains thereof is in particular given by a polypeptide comprising SEQ ID NO: 2, SEQ ID NO: 4, SEQ ID NO: 5, SEQ ID NO: 6, SEQ ID NO: 23, SEQ ID NO: 40, SEQ ID NO: 41, and SEQ ID NO: 42 or an amino acid sequence with at least 90%, 91%, 92%, 93%, 94%, or 95%, identity to any one of SEQ ID NOs: 2, 4, 5, 6, 23, and 40-42. In various embodiments, the binding affinity for the spike protein or domains thereof is given by a polypeptide having SEQ ID NO: 7, SEQ ID NO: 8, SEQ ID NO: 17, SEQ ID NOs: 43-47, SEQ ID NO: 66, or SEQ ID NO: 67, or an amino acid sequence with at least 90%, 91%, 92%, 93%, 94%, or 95%, identity to any one of SEQ ID NOs: 7, 8, 17, 43-47, 66, 67.

Before the present invention is described in more detail below it is to be understood that this invention is not limited to the particular methodology, protocols and reagents described herein as these may vary. It is also to be understood that the terminology used herein is for the purpose of describing particular aspects and embodiments only and is not intended to limit the scope of the present invention, which is reflected by the appended items. Unless defined otherwise, all technical and scientific terms used herein have the same meaning as commonly understood by one of ordinary skill in the art to which this invention belongs. This includes a skilled person working in the field of protein engineering and purification, but also including a skilled person working in the field of developing new specific binding molecules for the spike glycoprotein or domains thereof for use in technical applications such as affinity chromatography.

Preferably, the terms used herein are defined as described in “A multilingual glossary of biotechnological terms: (IUPAC Recommendations)”, Leuenberger, H. G. W, Nagel, B. and KoIbl, H. eds. (1995), Helvetica Chimica Acta, CH-4010 Basel, Switzerland). Throughout this specification and the items, which follow, unless the context requires otherwise, the word “comprise”, and variants such as “comprises” and “comprising”, will be understood to imply the inclusion of a stated integer or step, or group of integers or steps, but not the exclusion of any other integer or step or group of integers or steps. The term “comprise(s)” or “comprising” may encompass a limitation to “consists of” or “consisting of”, should such a limitation be necessary for any reason and to any extent.

Several documents (for example: patents, patent applications, scientific publications, manufacturer's specifications, instructions, GenBank Accession Number, etc.) may be cited throughout the present specification. Nothing herein is to be construed as an admission that the invention is not entitled to antedate such disclosure by virtue of prior invention. Some of the documents cited herein may be characterized as being “incorporated by reference”. In the event of a conflict between the definitions or teachings of such incorporated references and definitions or teachings recited in the present specification, the text of the present specification takes precedence.

All sequences referred to herein are disclosed in the accompanying sequence listing that, with its whole content and disclosure, forms part of the disclosure content of the present specification.

General Definitions of Important Terms Used in the Application

The term “SARS-Cov-2” as used herein, refers to the severe acute respiratory syndrome corona virus 2, or variants thereof, including mutants or related viruses.

The terms “spike protein” or “spike glycoprotein” or “surface glycoprotein” or “spike glycoprotein S” or “COVID-19” may be used interchangeably herein and refer to an amino acid sequence as shown in GenBank ID: QHD43416.1, or variants thereof. The term “spike protein” comprises all polypeptides which show an amino acid sequence identity of at least 70%, 80%, 85%, 90%, 95%, 96% or 97% or more, or 100% to GenBank ID: QHD43416.1 (SEQ ID NO: 10). As described herein, the term “spike protein” comprises, without being limited thereto, polypeptides of SARS-Cov-2 variants that emerged in the United Kingdom (“B.1.1.7”), South Africa (“B.1.351”), Brazil (“P.1”), and India (“B.1.617.2”). On May 31, 2021, the WHO (World Health Organization) has announced that these variants are named, according to WHO label, variant Alpha (“B.1.1.7”), Beta (“B.1.351”), Gamma (“P.1”), and Delta (“B.1.617.2”), respectively. The term “spike protein” also comprises polypeptides of SARS-Cov-2 variants named, according to the WHO label, Epsilon (“B.1.427/B.1.429”), Zeta (“P.2”), Eta (“B.1.525”), Theta (“P.3”), Iota (“B.1.526”), and Kappa (“B.1.617.1”).

In various embodiments, the binding proteins of the present invention may show binding affinity as described elsewhere herein for the spike protein of any of variants Alpha (“B.1.1.7”), Beta (“B.1.351”), Gamma (“P.1”), Delta (“B.1.617.2”), Epsilon (“B.1.427/B.1.429”), Zeta (“P.2”), Eta (“B.1.525”), Theta (“P.3”), Iota (“B.1.526”), and Kappa (“B.1.617.1”). In preferred embodiments, the binding proteins of the present invention show binding affinity as described elsewhere herein for the spike protein of variant Alpha (“B.1.1.7”) and/or variant Beta (“B.1.351”). In additional and/or other embodiments, the binding proteins of the present invention may or may not show binding affinity as described elsewhere herein for the spike protein of variant Delta (“B.1.617.2”). The term “S1 domain” or “51” or “51 subunit” or “51 protein” or “spike glycoprotein Subunit 1” or “S glycoprotein subunit 1” or “spike protein 51” as used herein, refers to any S1 domain of the spike glycoprotein or variants thereof. Amino acids at positions 16-685 of GenBank ID: QHD43416.1 correspond to the S1 domain of the spike protein or variant thereof. The term “S1 domain” comprises all polypeptides which show an amino acid sequence identity of at least 70%, 80%, 85%, 90%, 95%, 96% or 97% or more, or 100% to amino acids at positions 16-685 of GenBank ID: QHD43416.1 (SEQ ID NO: 11). The term “S1 domain” comprises in particular S1 domains of polypeptides of SARS-Cov-2 variants described above, more specifically variants Alpha (“B.1.1.7”), Beta (“B.1.351”), Gamma (“P.1”), Delta (“B.1.617.2”), Epsilon (“B.1.427/B.1.429”), Zeta (“P.2”), Eta (“B.1.525”), Theta (“P.3”), Iota (“B.1.526”), and Kappa (“B.1.617.1”), wherein the S1 domains of the respective SARS-Cov-2 variants essentially correspond with regard to their positions in the spike protein to positions 16-685 of the sequence of GenBank ID: QHD43416.1, or essentially correspond to the sequence of the S1 domain shown in SEQ ID NO: 11.

The term “receptor binding domain” or “RBD” or “SARS-CoV-52 S protein RBD” or “S protein RBD” or “spike glycoprotein receptor-binding domain” or “spike protein RBD” is part of the S1 domain of the spike protein and corresponds to amino acids at positions 319-541 of GenBank ID: QHD43416.1. The term “RBD” comprises all polypeptides which show an amino acid sequence identity of at least 70%, 80%, 85%, 90%, 95%, 96% or 97% or more, or 100% to amino acids 319-541 of GenBank ID: QHD43416.1 (SEQ ID NO: 12). The term “RBD” comprises in particular RBDs of polypeptides of SARS-Cov-2 variants described above, more specifically variants Alpha (“B.1.1.7”), Beta (“B.1.351”), Gamma (“P.1”), Delta (“B.1.617.2”), Epsilon (“B.1.427/B.1.429”), Zeta (“P.2”), Eta (“B.1.525”), Theta (“P.3”), Iota (“B.1.526”), and Kappa (“B.1.617.1”) wherein the RBDs of the respective SARS-Cov-2 variants essentially correspond with regard to their positions in the S1 domain to positions 319-541 of the sequence of GenBank ID: QHD43416.1, or essentially correspond to the sequence of the RBD shown in SEQ ID NO: 12.

The term . . . binding protein for the spike protein or domains thereof” or “affinity ligand for the spike protein or domains thereof” describes a protein that is capable to bind to the spike protein and/or the S1 domain of the spike glycoprotein and/or the RBD as described herein, including the S1 domains and/or RBDs of polypeptides of SARS-Cov-2 variants described herein. As described herein, a “binding protein for the spike protein or domains thereof” or “affinity ligand for the spike protein or domains thereof” refers to a protein with detectable interaction with the spike protein domain and/or the S1 domain and/or the RBD, as determined by suitable methods such as for example SPR analysis or any other appropriate technology known to someone skilled in the art. The binding protein for the spike protein may bind to the RBD. A binding protein for the spike protein may bind to S1 domain. A binding protein for the spike protein may bind to a particle, for example a virus particle, comprising a spike protein and/or S1 domain and/or RBD. Preferably, the binding affinity is 500 nM for the spike protein, and/or 500 nM for the S1 domain, and/or 500 nM for the RBD. More preferably, the binding affinity is 100 nM for the spike protein, and/or 100 nM for the 51 domain, and/or 100 nM for the RBD.

The terms “binding affinity” and “binding activity” may be used herein interchangeably and they refer to the ability of a polypeptide of the invention to bind to another protein, peptide, or fragment or domain thereof. Binding affinity is typically measured and reported by the equilibrium dissociation constant (KD) (usually measured in “mol/L”, sometimes abbreviated as “M”), which is used to evaluate and rank the strength of bimolecular interactions in particular the interaction between a first protein and a second protein. The binding affinity and dissociation constants can be measured quantitatively. Methods for determining binding affinities are well known to the skilled person and can be selected, for instance, from the following methods that are well established in the art: surface plasmon resonance (SPR) spectroscopy, enzyme-linked immunosorbent assay (ELISA), kinetic exclusion analysis (KinExA assay), Bio-layer interferometry (BLI), flow cytometry, fluorescence spectroscopy techniques, isothermal titration calorimetry (ITC), analytical ultracentrifugation, radioimmunoassay (RIA or IRMA), and enhanced chemiluminescence (ECL). Typically, the dissociation constant KD is determined at temperatures in the range of 20° C. and 30° C. If not specifically indicated otherwise, KD values recited herein are determined at 25° C. by SPR. The most widely used SPR-based system is the BIAcore, produced by BIAcore AB. In various embodiments of the present invention, the binding affinity for SARS-Cov-2 may be determined by the BIAcore SPR system.

The term “fusion protein” relates to a protein comprising at least a first protein joined genetically to at least a second protein. A fusion protein is created through joining of two or more genes that originally coded for separate proteins. Thus, a fusion protein may comprise a multimer of identical or different proteins which are expressed as a single, linear polypeptide.

As used herein, the term “linker” refers in its broadest meaning to a molecule that covalently joins at least two other molecules.

The term “amino acid sequence identity” refers to a quantitative comparison of the identity (or differences) of the amino acid sequences of two or more proteins. “Percent (%) amino acid sequence identity” with respect to a reference polypeptide sequence is defined as the percentage of amino acid residues in a sequence that are identical with the amino acid residues in the reference polypeptide sequence, after aligning the sequences and introducing gaps, if necessary, to achieve the maximum percent sequence identity. To determine the sequence identity, the sequence of a query protein is aligned to the sequence of a reference protein or polypeptide, for example, to the polypeptide of SEQ ID NO: 1. Methods for sequence alignment are well known in the art. For example, for determining the extent of an amino acid sequence identity of an arbitrary polypeptide relative to the amino acid sequence of, for example, SEQ ID NO: 1, the SIM Local similarity program is preferably employed (Xiaoquin Huang and Webb Miller (1991), Advances in Applied Mathematics, vol. 12: 337-357), that is freely available. For multiple alignment analysis, ClustalW is preferably used (Thompson et al. (1994) Nucleic Acids Res., 22(22): 4673-4680).

The terms “protein” and “polypeptide” refer to any chain of two or more amino acids linked by peptide bonds and does not refer to a specific length of the product. Thus, “peptides”, “protein”, “amino acid chain”, or any other term used to refer to a chain of two or more amino acids, are included within the definition of “polypeptide”, and the term “polypeptide” may be used instead of, or interchangeably with, any of these terms. The term “polypeptide” is also intended to refer to the products of post-translational modifications of the polypeptide like, e.g., glycosylation, which are well known in the art.

The term “alkaline stable” or “alkaline stability” or “caustic stable” or “caustic stability” refers to the ability of the binding protein for the spike protein to withstand alkaline conditions without significantly losing the ability to bind to the S1 domain of the spike protein of SARS-Cov-2. The skilled person in this field can test alkaline stability by incubating binding protein for the spike protein with sodium hydroxide solutions, e.g., as described in the Examples, and subsequent testing of the binding activity to the spike protein S1 by routine experiments known to someone skilled in the art, for example, by chromatographic approaches. The alkaline stability may be determined by coupling a spike protein binding protein of the invention to a surface plasmon resonance (SPR) sensor chip, and assaying the binding capacity or binding activity for the S1 domain before and after exposure to an alkaline solution. The alkaline treatment may be performed, for instance, in 0.1 M NaOH for an extended period of time, e.g., at least 10 h, at room temperature (22° C.+/−3° C.). As further described herein, binding proteins of the invention, in particular fusion proteins, may retain at least 70% binding affinity for the S1 domain or RBD after exposure to alkaline conditions as described herein. In various embodiments, the binding proteins of the invention retain binding affinity for the S1 domain or RBD as described above when immobilized to a solid support, preferably to a solid support of an affinity separation matrix.

The term “chromatography” refers to separation technologies which employ a mobile phase and a stationary phase to separate one type of molecules (e.g., the spike protein or a protein comprising a S1 domain or a protein comprising a RBD) from other molecules (e.g., contaminants) in the sample. The liquid mobile phase contains a mixture of molecules and transports these across or through a stationary phase (such as a solid matrix). Due to the differential interaction of the different molecules in the mobile phase with the stationary phase, molecules in the mobile phase can be separated. The term “affinity chromatography” refers to a specific mode of chromatography in which a ligand (i.e. a binding protein for the spike protein or domains thereof) coupled to a stationary phase interacts with a molecule (i.e. protein comprising spike protein or a domain thereof) in the mobile phase (the sample) i.e. the ligand has a specific binding affinity for the molecule to be purified. As understood in the context of the invention, affinity chromatography involves the addition of a sample containing a protein comprising spike protein or a domain thereof to a stationary phase which comprises a chromatography ligand, such as a binding protein for the spike protein. The terms “solid support” or “solid matrix” are used interchangeably for the stationary phase. The terms “affinity matrix” or “affinity purification matrix” or “affinity chromatography matrix”, as used interchangeably herein, refer to a matrix, e.g., a chromatographic matrix, onto which an affinity ligand e.g., a binding protein for the spike protein or a domain thereof is attached. The attached affinity ligand (e.g., binding protein for the spike protein or a domain thereof) is capable of specific binding to a molecule of interest (e.g., SARS-Cov-2 or variants thereof or related viruses) which is to be purified or removed from a mixture (e.g., in a liquid sample). The term “affinity purification” as used herein refers to a method of purifying a protein comprising spike protein or a domain thereof from a liquid (sample) by binding the protein or particle comprising spike protein or a domain thereof to binding protein for the spike protein or a domain thereof that is immobilized to a matrix. Thereby, other components of the mixture except protein or particle comprising S1 are removed. In a further step, the bound protein or particle comprising the spike protein or a domain thereof can be eluted and obtained in highly purified form.

### Detailed Description of Embodiments of the Invention

The present invention will now be further described. In the following passages different aspects of the invention are defined in more detail. Each aspect defined below may be combined with any other aspect or aspects unless clearly indicated to the contrary. In particular, any feature indicated as being preferred or advantageous may be combined with any other feature or features indicated as being preferred or advantageous.

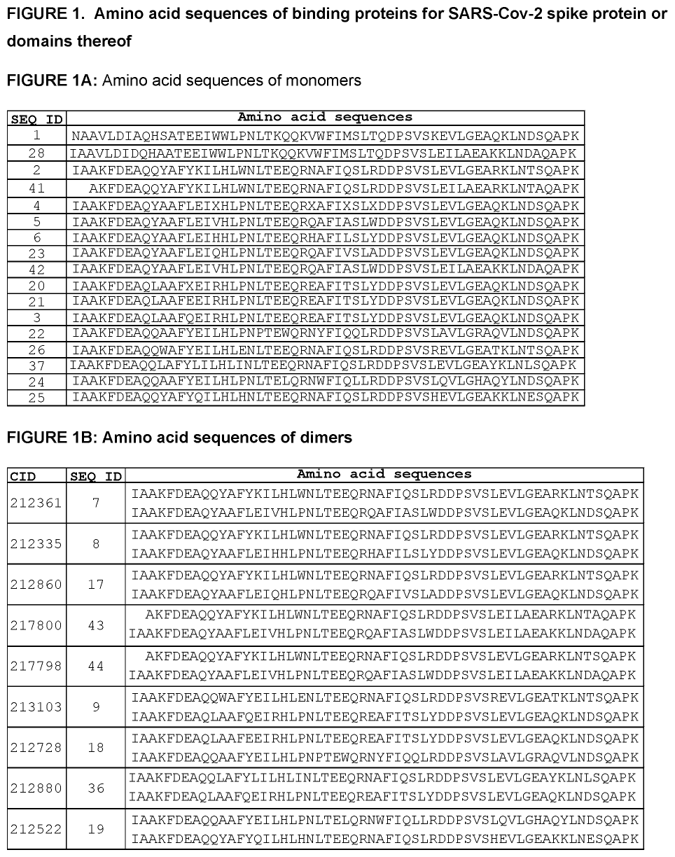

The novel binding protein for the spike protein or a domain thereof exhibit a high binding affinity for the spike protein or a domain thereof of SARS-Cov-2 or variants thereof described herein. The binding protein for the spike protein or a domain thereof comprises an amino acid sequence selected from the group SEQ ID NO: 1, SEQ ID NO: 2, SEQ ID NO: 3, SEQ ID NO: 4, SEQ ID NO: 5, or SEQ ID NO: 6, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 37, or SEQ ID NOs: 40-42, or an amino acid with at least 80%, preferably at least 90%, 91%, 92%, 93%, 94%, or 95%, sequence identity to any one of SEQ ID NOs: 1-6, 20-28, 37, and 40-42. In some embodiments, a binding protein for the spike protein or a domain thereof is comprising at least one amino acid sequence as shown in FIG. 1 or variants thereof as described herein. The binding affinity for the spike protein or domains thereof is in particular given by a polypeptide comprising SEQ ID NO: 2, SEQ ID NO: 4, SEQ ID NO: 5, SEQ ID NO: 6, SEQ ID NO: 23, SEQ ID NO: 40, SEQ ID NO: 41, or SEQ ID NO: 42, or an amino acid sequence with at least 92%, or at least 93%, identity to any one of SEQ ID NOs: 2, 4, 5, 6, 23, 40-42.

In some embodiments, the binding protein for the spike protein has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to any one of the amino sequences of SEQ ID NOs: 2, 4-8, 17, 23, 40-42, 43-45, 66 and 67. In various other embodiments described herein referring to at least 85% sequence identity, the sequence identity may preferably be any of at least 90%, 91%, 92%, 93%, 94%, or 95%, identity to the respective reference sequence.

In some embodiments, the binding protein for the spike protein or a domain thereof is comprising an amino acid sequence with at least 85% sequence identity to SEQ ID NO: 1: NAAVLDIAQHSATEEIVWVLPNLTKQQKVWFIMSLTQDPSVSKEVLGEAQKLNDSQAPK.

In some embodiments, the binding protein for the spike protein or a domain thereof is comprising an amino acid sequence with at least 85% sequence identity, preferably at least 92% sequence identity, to at least 56 amino acids of SEQ ID NO: 2: IAAKFDEAQQYAFYKILHLWNLTEEQRNAFIQSLRDDPSVSLEVLGEARKLNTSQAPK.

Considering SEQ ID NO: 2 and related dimers according to SEQ ID NOs: 7, 8, 17, 43-47, 66, and 67, in various embodiments of the binding proteins of the invention, the amino acid at the position corresponding to position 11 in SEQ ID NO: 15 is not A. In various embodiments, in the binding proteins of the invention, the amino acid at the position corresponding to position 11 in SEQ ID NO: 2 or SEQ ID NO: 15 is Y.

Accordingly, in various embodiments, in the binding proteins of the invention, the amino acid at the position corresponding to position 15 in SEQ ID NO: 15 is not E. In various embodiments, in the binding proteins of the invention, the amino acid at the position corresponding to position 15 in SEQ ID NO: 2 or SEQ ID NO: 15 is K.

Further, in various embodiments, in the binding proteins of the invention, the amino acid at the position corresponding to position 20 in SEQ ID NO: 15 is not P. In various embodiments, in the binding proteins of the invention, the amino acid at the position corresponding to position 20 in SEQ ID NO: 2 or SEQ ID NO: 15 is W. In preferred embodiments, in the first (or N-terminally located) monomer of dimeric or multimeric binding proteins of the invention, the amino acid at the position corresponding to position 20 in SEQ ID NO: 15 is not P. In preferred embodiments, in the first (or N-terminal) monomer of dimeric or multimeric binding proteins of the invention, the amino acid at the position corresponding to position 20 in SEQ ID NO: 2 or SEQ ID NO: 15 is W. Further, in various embodiments, in the binding proteins of the invention, the amino acid at the position corresponding to position 49 in SEQ ID NO: 15 is not Q. In various embodiments, in the binding proteins of the invention, the amino acid at the position corresponding to position 49 in SEQ ID NO: 2 or SEQ ID NO: 15 is R.

Further, in various embodiments, in the binding proteins of the invention, the amino acid at the position corresponding to position 53 in SEQ ID NO: 15 is not D. In various embodiments, in the binding proteins of the invention, the amino acid at the position corresponding to position 53 in SEQ ID NO: 2 or SEQ ID NO: 15 is T.

Further, in various embodiments, in the binding proteins of the invention, the amino acid at the position corresponding to position 42 in SEQ ID NO: 2 or SEQ ID NO: 15 is L.

In various embodiments, in the binding proteins of the invention, the amino acid at the position corresponding to position 11, 15, 20, 49, and 53, in SEQ ID NO: 15 is not A, E, P, Q, and D, respectively. In various embodiments, in the binding proteins of the invention, the amino acid at the position corresponding to position 11, 15, 20, 49, and 53, in SEQ ID NO: 2 or SEQ ID NO: 15 is Y, K, W, R, and T, respectively.

Further, in various embodiments, in the binding proteins of the invention, the amino acid at the position corresponding to position 11, 15, 20, 42, 49, and 53, in SEQ ID NO: 2 or SEQ ID NO: 15 is Y, K, W, L, R, and T, respectively.

In various embodiments, considering SEQ ID NOs: 4, 5, 6, 23, and 42, and related dimers according to SEQ ID NOs: 7, 8, 17, 43-47, 66, and 67, in the second (or C-terminally located) monomer of a binding protein of the invention, the amino acid at the position corresponding to position 10 in SEQ ID NO: 15 is not Q. In various embodiments, in the binding proteins of the invention, the amino acid at the position corresponding to position 10 in SEQ ID NO: 4, 5, 6, 23, and 42, or SEQ ID NO: 15, is Y.

Accordingly, in various embodiments, in the second (or C-terminally located) monomer of a binding protein of the invention, the amino acid at the position corresponding to position 14 in SEQ ID NO: 15 is not Y. In various embodiments, in the binding proteins of the invention, the amino acid at the position corresponding to position 14 in SEQ ID NO: 4, 5, 6, 23, and 42, or SEQ ID NO: 15, is L.

Further, in various embodiments, in the second (or C-terminally located) monomer of a binding protein of the invention, the amino acid at the position corresponding to position 17 in SEQ ID NO: 15 is not L. In various embodiments, in the binding proteins of the invention, the amino acid at the position corresponding to position 17 in SEQ ID NO: 4, 5, 6, 23, and 42, or SEQ ID NO: 15, is V, H, or Q.

Further, in various embodiments, in the second (or C-terminally located) monomer of a binding protein of the invention, the amino acid at the position corresponding to position 28 in SEQ ID NO: 15 is not N. In various embodiments, in the binding proteins of the invention, the amino acid at the position corresponding to position 28 in SEQ ID NO: 4, 5, 6, 23, and 42, or SEQ ID NO: 15, is Q, or H.

Further, in various embodiments, in the second (or C-terminally located) monomer of a binding protein of the invention, the amino acid at the position corresponding to position 32 in SEQ ID NO: 15 is not Q. In various embodiments, in the binding proteins of the invention, the amino acid at the position corresponding to position 32 in SEQ ID NO: 4, 5, 6, 23, and 42, or SEQ ID NO: 15, is a hydrophobic amino acid, preferably A, L, or V.

Further, in various embodiments, in the second (or C-terminally located) monomer of a binding protein of the invention, the amino acid at the position corresponding to position 35 in SEQ ID NO: 15 is not R. In various embodiments, in the binding proteins of the invention, the amino acid at the position corresponding to position 35 in SEQ ID NO: 4, 5, 6, 23, and 42, or SEQ ID NO: 15, is W, Y, or A.

In various embodiments, in the second (or C-terminally located) monomer of a binding protein of the invention, the amino acid at the position corresponding to position 10, 11, 14, 15, 17, 20, 28, 32, 35, 49, and 53, in SEQ ID NO: 4, 5, 6, 23, and 42, or SEQ ID NO: 15, is Y, Y, L, K, (V, H, or Q), W, (Q or H), (A, L, or V), (W, Y, or A), R, and T, respectively. Furthermore, in various embodiments, in the second (or C-terminally located) monomer of a binding protein of the invention, the amino acid at the position corresponding to position 10, 11, 14, 15, 17, 20, 28, 32, 35, 42, 49, and 53, in SEQ ID NO: 4, 5, 6, 23, and 42, or SEQ ID NO: 15, is Y, Y, L, K, (V, H, or Q), W, (Q or H), (A, L, or V), (W, Y, or A), L, R, and T, respectively. In various embodiments, a binding protein of the invention has, in comparison to the cs27 sequence of SEQ ID NO: 15, at least 5 or 6 amino acid substitutions at positions corresponding to any of positions 10, 11, 14, 15, 17, 20, 28, 32, 35, 49, and 53 in the sequence of SEQ ID NO: 15. In various embodiments, a binding protein of the invention has, in comparison to the cs27 sequence of SEQ ID NO: 15, at least 5 or 6 amino acid substitutions at positions corresponding to any of positions 10, 11, 14, 15, 17, 20, 28, 32, 35, 49, and 53 in the sequence of SEQ ID NO: 15, and does not have a substitution at the position corresponding to position 42 in SEQ ID NO: 15.

In various embodiments, a binding protein of the invention has, in comparison to the cs27 sequence of SEQ ID NO: 15, 5 amino acid substitutions at positions corresponding to positions 11, 15, 20, 49, and 53 in the sequence of SEQ ID NO: 15. In various embodiments, a binding protein of the invention has, in comparison to the cs27 sequence of SEQ ID NO: 15, 5 amino acid substitutions at positions corresponding to positions 11, 15, 20, 49, and 53 in the sequence of SEQ ID NO: 15, and does not have a substitution at the position corresponding to position 42 in SEQ ID NO: 15.

In various embodiments, a binding protein of the invention has, in comparison to the cs27 sequence of SEQ ID NO: 15, at least 5 or 6 amino acid substitutions at positions corresponding to any of positions 10, 11, 14, 15, 17, 20, 28, 32, 35, 49, and 53 in the sequence of SEQ ID NO: 15, wherein the substitutions are selected from any of Q10Y, A11Y, Y14L, E15K, L17(V, H, or Q), P20W, N28(Q or H), Q32(A, L, or V), R35(W, Y, or A), Q49R, and D53T. In various embodiments, a binding protein of the invention has, in comparison to the cs27 sequence of SEQ ID NO: 15, at least 5 or 6 amino acid substitutions at positions corresponding to any of positions 10, 11, 14, 15, 17, 20, 28, 32, 35, 49, and 53 in the sequence of SEQ ID NO: 15, and does not have a substitution at the position corresponding to position 42 in SEQ ID NO: 15, wherein the substitutions are selected from any of Q10Y, A11Y, Y14L, E15K, L17(V, H, or Q), P20W, N28(Q or H), Q32(A, L, or V), R35(W, Y, or A), Q49R, and D53T.

In various embodiments, a binding protein of the invention has, in comparison to the cs27 sequence of SEQ ID NO: 15, 5 amino acid substitutions at positions corresponding to any of positions 11, 15, 20, 49, and 53 in the sequence of SEQ ID NO: 15, wherein the substitutions are selected from any of A11Y, E15K, P20W, Q49R, and D53T.

In various embodiments, a binding protein of the invention has, in comparison to the cs27 sequence of SEQ ID NO: 15, 5 amino acid substitutions at positions corresponding to any of positions 11, 15, 20, 49, and 53 in the sequence of SEQ ID NO: 15, and does not have a substitution at the position corresponding to position 42 in SEQ ID NO: 15, wherein the substitutions are selected from any of A11Y, E15K, P20W, Q49R, and D53T.

In various embodiments, positions corresponding to positions 1 and 2 of the cs27 sequence of SEQ ID NO: 15 may be deleted in a binding protein of the invention, as illustrated, e.g., in SEQ ID NOs: 40 and 41.

In further embodiments, in the binding proteins of the invention, the amino acids at the positions corresponding to positions 44, 46, and/or 54 in SEQ ID NO: 15 may not be V, G, and S, respectively, as illustrated in SEQ ID NO: 41. In various embodiments, in the binding proteins of the invention, the amino acids at the positions corresponding to positions 44, 46, and/or 54 in SEQ ID NO: 15 are I, A, and A, respectively, as illustrated in positions 42, 44, and 52 of SEQ ID NO: 41. In various embodiments, the binding protein of the present invention may have at least 92%, at least 93%, or at least 94% sequence identity to the sequence of SEQ ID NO: 2 or the sequence of SEQ ID NO: 40 or SEQ ID NO: 41. In such embodiments, the amino acid at the position corresponding to position 49 in SEQ ID NO: 15 may be K, as illustrated in position 49 of SEQ ID NO: 42.

In various embodiments, the binding protein of the present invention may have at least 92% sequence identity to the sequence of SEQ ID NO: 5.

In some embodiments, the binding protein for the spike protein or a domain thereof is comprising an amino acid sequence with at least 85%, preferably 92%, sequence identity to SEQ ID NO: 4:

IAAKFDEAQYAAFLEIX₁HLPNLTEEQRX₂AFIX₃SLX₄DDPSVSLEVLG
EAQKLNDSQAPK,

wherein individually of each other, X 1 is selected from any amino acid, preferably H or V or Q, X₂ is selected from any amino acid, preferably H or Q, X₃ is selected from any amino acid, preferably a hydrophobic amino acid, preferably A or L or V, and X₄ is selected from an aromatic amino acid, preferably W or Y, or A.

In some embodiments, the binding protein for the spike protein or a domain thereof is comprising an amino acid sequence with at least 85%, preferably 92%, sequence identity to SEQ ID NO: 5:

IAAKFDEAQYAAFLEIVHLPNLTEEQRQAFIASLWDDPSVSLEVLGEAQ
KLNDSQAPK

In some embodiments, the binding protein for the spike protein or a domain thereof is comprising an amino acid sequence with at least 85%, preferably 92%, sequence identity to SEQ ID NO: 6:

IAAKFDEAQYAAFLEIHHLPNLTEEQRHAFILSLYDDPSVSLEVLGEAQ
KLNDSQAPK.

In some embodiments, the binding protein for the spike protein or a domain thereof is comprising an amino acid sequence with at least 85%, preferably 92%, sequence identity to SEQ ID NO: 23.

SEQ ID NOs: 5, 6, 23, and 42, illustrate the following motifs for the positions corresponding to positions 10, 14, 17, 28, 32, and 35 in the consensus sequence of SEQ ID NO: 4: YLVQAW (SEQ ID NOs: 5 and 42), YLHHLY (SEQ ID NO: 6), and YLQQVA (SEQ ID NO: 23). Preferred embodiments of the present invention pertaining to SEQ ID NOs: 5, 6, 23, and 42, in particular the second (or C-terminally located) monomer of (hetero-)dimers of the invention, may include an I (lie) at the position corresponding to position 44 of SEQ ID NO: 4, and an A (Ala) at the positions corresponding to positions 46 and 54 of SEQ ID NO: 4. Other preferred embodiments of the present invention pertaining to SEQ ID NOs: 5, 6, 23, and 42, in particular the second (or C-terminally located) monomer of (hetero-)dimers, may include an I (lie) at the position corresponding to position 44 of SEQ ID NO: 4, a K (Lys) at the position corresponding to position 49 of SEQ ID NO: 4, and an A (Ala) at the position corresponding to positions 46 and 54 of SEQ ID NO: 4, as illustrated, e.g., in SEQ ID NO: 42.

In some embodiments, the binding protein for the spike protein or a domain thereof is comprising an amino acid sequence with at least 85% sequence identity, preferably at least 92% sequence identity, to at least 56 amino acids of SEQ ID NO: 20:

IAAKFDEAQLAAFXEIRHLPNLTEEQREAFITSLYDDPSVSLEVLGEAQ
KLNDSQAPK,

wherein X is selected from any amino acid. In some embodiments, X may be selected from Q or E. SEQ ID NO: 20 illustrates the following motif for the positions corresponding to positions 10, 14, 17, 28, 32, and 35 in the sequence of SEQ ID NO: 4: LXRETY. Accordingly, in preferred embodiments of the present invention pertaining to SEQ ID NO: 20, including the first (or N-terminally located) or second (or C-terminally located) monomer of (hetero-)dimers of the invention, the positions corresponding to positions 10, 14, 17, 28, 32, and 35 in the sequence of SEQ ID NO: 4 are L, X, R, E, T, and Y, respectively, wherein X may be any amino acid, preferably Q or E as illustrated in SEQ ID NOs: 3 and 21, respectively. In some embodiments, the binding protein for the spike protein or a domain thereof is comprising an amino acid sequence with at least 85% sequence identity, preferably at least 92% sequence identity, to at least 56 amino acids of SEQ ID NO: 21. In some embodiments, the binding protein for the spike protein or a domain thereof is comprising an amino acid sequence with at least 85% sequence identity, preferably at least 92% sequence identity, to at least 56 amino acids of SEQ ID NO: 3:

IAAKFDEAQLAAFQEIRHLPNLTEEQREAFITSLYDDPSVSLEVLGEAQ
KLNDSQAPK.

In some embodiments, the binding protein for the spike protein or a domain thereof is comprising an amino acid sequence with at least 85% sequence identity, preferably at least 92% sequence identity, to SEQ ID NO: 22, or to at least 56 amino acids of SEQ ID NO: 22. In some embodiments, the binding protein for the spike protein or a domain thereof is comprising an amino acid sequence with at least 85%, preferably at least 92%, sequence identity to SEQ ID NO: 37. In some embodiments, the binding protein for the spike protein or a domain thereof is comprising an amino acid sequence with at least 85%, preferably at least 92%, sequence identity to SEQ ID NO: 24. In some embodiments, the binding protein for the spike protein or a domain thereof is comprising an amino acid sequence with at least 85%, preferably at least 92%, sequence identity to SEQ ID NO: 25. In some embodiments, the binding protein for the spike protein or a domain thereof is comprising an amino acid sequence with at least 85%, preferably at least 92%, sequence identity, to SEQ ID NO: 28:

AVLDIDQHAATEEIWWLPNLTKQQKVWFIMSLTQDPSVSLEILAEAKKL
NDAQAPK.

One advantage of the disclosed binding protein for the spike protein is the important functional characteristic that it binds specifically to the spike protein or to a domain thereof. Needless to point out, that this is of particular advantage in the purification of proteins or particles comprising spike protein or a domain thereof, for example, vaccines specific for SARS-Cov-2. The binding protein for the S1 domain is functionally characterized by a binding affinity, preferably of less than 500 nM for the S1 domain of SARS-Cov-2. In some embodiments, the binding protein for the spike protein binds to spike protein or a domain thereof, preferably with a dissociation constant KD below 50 nM, or more preferably below 10 nM, as shown in Example 3.

The binding protein for the S1 domain binds highly specific to the S1 domain or RBD of SARS-Cov2. In some embodiments, the binding proteins of the invention bind highly specific to the S1 domain or RBD of variants of SARS-Cov-2, for example, to the British variant Alpha (“B.1.1.7”). In some embodiments, the binding protein of the invention binds to the South African variant Beta (“B.1.351”). In some embodiments, the binding protein of the invention binds to the Brazilian variant Gamma (“P.1”). No significant binding affinity to the S1 domain or RBD for severe acute respiratory syndrome corona virus 1 (SARS-Cov-1), could be observed. No significant binding affinity to the S1 domain or RBD of Middle East Respiratory Syndrome (MERS) Coronavirus (MERS-Cov) could be observed.

A common structural feature of the binding proteins is that they are based on artificial mosaic proteins that are stable under conditions as usually applied in affinity chromatography, for example, under alkaline conditions. For example, the general scaffold of the SARS Cov-2 S1 protein binding protein is a triple helical scaffold of at least 56 amino acids based on SEQ ID NO: 15. The artificial mosaic proteins are characterized by a unique and surprising SARS Cov-2 spike protein binding functionality. SEQ ID NO: 16 (C27) or SEQ ID NO: 15 (cs27) do not bind to SARS Cov-2. More specifically, SEQ ID NO: 15 or SEQ ID NO: 16 does not bind to SARS Cov-2 spike protein, and has in particular been shown not to bind to the S1 domain or the RBD.

Multimers. In one embodiment of the invention, the binding protein for the spike protein comprises 1, 2, 3, 4, 5, or 6 binding protein(s) linked to each other. Multimers (including dimers) of the binding protein are generated artificially, generally by recombinant DNA technology well-known to a skilled person. In some embodiments, the multimer is a homo-multimer, e.g. the amino acid sequences of binding protein for the spike protein are identical. For example, the homo multimeric binding protein for the spike protein may comprise at least two monomers as shown in SEQ ID NO: 1 (or at least 92% identical thereto) linked to each other in head-to-tail orientation. For example, the homo-multimeric binding protein for the spike protein may comprise at least two monomers as shown in SEQ ID NO: 28 (or at least 92% identical thereto) linked to each other in head-to-tail orientation.

In other embodiments, the multimer is a hetero-multimer, e.g. the amino acid sequences of the binding protein for the spike protein are different. According to preferred embodiments, the multimer or hetero-multimer is a (hetero)-dimer. In preferred embodiments, a (hetero)-dimer of the invention lacks the two N-terminal amino acids corresponding to the amino acids at positions 1 and 2 of any of SEQ ID NOs: 2-6, 20-26, 28, 37, and 42. As described herein, the monomers of a multimer or hetero-multimer, preferably (hetero)-dimer, of the present invention may be linked to each other in head-to-tail orientation. As further described herein, in a (hetero)-dimer, the first monomer may be considered as the N-terminal monomer or the N-terminally located monomer, and the second monomer may be considered as the C-terminal monomer or the C-terminally located monomer.

In some embodiments, at least one monomer of the spike protein binding multimer has amino acid substitutions at any of positions corresponding to positions 10, 11, 14, 15, 17, 20, 28, 32, 35, 49, and/or 53 in the sequence of SEQ ID NO: 15, as described elsewhere herein. Optionally, position 1 and position 2 in the monomer are deleted. For example, the hetero-multimeric binding protein for the spike protein may comprise at least one monomer of SEQ ID NO: 2 or SEQ ID NO: 40. In some embodiments, a hetero-multimeric protein is a dimer comprising at least one monomer of SEQ ID NO: 2 (or SEQ ID NO: 40) at the N-terminus. For example, the hetero-multimeric binding protein for the spike protein may comprise SEQ ID NO: 2 (or SEQ ID NO: 40) and SEQ ID NO: 5 linked to each other in head-to-tail orientation. In some embodiments, the hetero-dimeric binding protein for the spike protein may comprise SEQ ID NO: 2 (or SEQ ID NO: 40) and has at least 85%, preferably at least 92% sequence identity to the amino sequence of SEQ ID NO: 7 (212361):

IAAKFDEAQQYAFYKILHLWNLTEEQRNAFIQSLRDDPSVSLEVLGEAR
KLNTSQAPK
IAAKFDEAQYAAFLEIVHLPNLTEEQRQAFIASLWDDPSVSLEVLGEAQ
KLNDSQAPK.

The first (N-terminal) monomer of the dimeric spike protein binding protein as shown in SEQ ID NO: 7 has substitutions A11Y, E15K, P20W, Q49R, D53T corresponding to SEQ ID NO: 15; and the second (C-terminal) monomer has substitutions Q10Y, Y14L, L17V, N28Q, Q32A, R35W corresponding to SEQ ID NO: 15.

In various embodiments, the first monomer of a dimeric spike protein binding protein of the invention has 5 substitutions in the first monomer as compared to the cs27 sequence of SEQ ID NO: 15, and has 6 substitutions in the second monomer as compared to the cs27 sequence of SEQ ID NO: 15. Preferably, the first monomer of a dimeric spike protein binding protein of the invention has 5 substitutions in the first monomer at positions corresponding to positions 11, 15, 20, 49, and 53 of SEQ ID NO: 15, and has 6 substitutions in the second monomer at positions corresponding to positions 10, 14, 17, 28, 32, and 35 of SEQ ID NO: 15. In various embodiments, the amino acids at the positions corresponding to said positions 10, 11, 14, 15, 17, 20, 28, 32, 35, 49, and 53, in SEQ ID NO: 15 are Y, Y, L, K, (V, H, or Q), W, (Q or H), (A, L, or V), (W, Y, or A), R, and T, respectively. In various embodiments, in the first and/or second monomer, the amino acid at the position corresponding to position 42 in SEQ ID NO: 15 is L.

In various embodiments, positions corresponding to positions 1 and 2 of the cs27 sequence of SEQ ID NO: 15 may be deleted in the first and/or second monomer.

For example, the hetero-multimeric binding protein for the spike protein may comprise SEQ ID NO: 2 and SEQ ID NO: 6 linked to each other in head-to-tail orientation. In some embodiments, the hetero-dimeric binding protein for the spike protein may comprise SEQ ID NO: 2 and has at least 85% sequence identity to the amino sequence of SEQ ID NO: 8 (212335):

IAAKFDEAQQYAFYKILHLWNLTEEQRNAFIQSLRDDPSVSLEVLGEAR
KLNTSQAPKIAAKF
DEAQYAAFLEIHHLPNLTEEQRHAFILSLYDDPSVSLEVLGEAQKLNDS
QAPK.

For example, the hetero-multimeric (for example dimer) binding protein for the spike protein may comprise SEQ ID NO: 2 and SEQ ID NO: 23 linked to each other in head-to-tail orientation. In some embodiments, the binding protein for the spike protein or a domain thereof may comprise SEQ ID NO: 2 and has at least 85%, sequence identity to the amino sequence of SEQ ID NO: 17 (212860).

Some embodiments refer to multimeric binding proteins for spike protein having at least 92%, at least 94%, at least 95%, at least 96%, at least 97%, at least 98%, or at least 99% amino acid sequence identity to the amino acid sequence selected from the group of one of SEQ ID NO: 7, 8, 17, 43-47, 66, 67. Some embodiments refer to SEQ ID NO: 7, 8, 17, 43-47, 66, 67 as multimeric spike protein binding proteins.

In some embodiments, the N-terminal monomers of the dimers of SEQ ID NO: 7, 8, 17, and 44 are identical (see FIG. 1B). In some embodiments, the C-terminal monomers of the dimers of SEQ I D NO: 7, 8, and 17 have different substitutions in four positions corresponding to positions 17, 28, 32, and 35 of SEQ ID NO: 15. The dimeric amino acid sequences of SEQ ID NO: 7, 8, and 17 share at least 96% sequence identity with each other. In some embodiments, the binding protein for the spike protein of severe acute respiratory syndrome corona virus 2 (SARS-Cov-2) is a multimer, preferably a dimer, that comprises an amino acid sequence with at least 92% sequence identity, to any one selected from the group of SEQ ID NOs: 2, 40, 41 linked in head-to-tail orientation to an amino acid sequence with at least at least 92% sequence identity, to any one selected from the group of SEQ ID NOs: 4, 5, 6, 23, and 42. Preferably, the binding protein has a binding affinity of less than 500 nM for the spike protein or domains thereof.

The present invention encompasses a hetero-dimeric binding protein comprising a first (N-terminal) monomer having at least 90%, 91%, 92%, 93%, 94%, or 95%, sequence identity to any of SEQ ID NOs: 2, 40, 41, preferably SEQ ID NO: 41, and a second (C-terminal) monomer having at least 90%, 91%, 92%, 93%, 94%, or 95%, sequence identity to any of SEQ ID NOs: 4, 5, 6, 23, and 42, preferably SEQ ID NO: 5, more preferably SEQ ID NO: 42. Preferably, the binding protein has a binding affinity of less than 500 nM, more preferably less than 100 nM, for the spike protein or domains thereof.

In preferred embodiments, a hetero-dimeric binding protein comprising a first (N-terminal) monomer having at least 90%, 91%, 92%, 93%, 94%, or 95%, sequence identity to any of SEQ ID NOs: 2, 40, 41, preferably SEQ ID NO: 41, and a second (C-terminal) monomer having at least 90%, 91%, 92%, 93%, 94%, or 95%, sequence identity to any of SEQ ID NOs: 4, 5, 6, 23, and 42, preferably SEQ ID NO: 5, more preferably SEQ ID NO: 42, has in the first (N-terminal) monomer a tyrosine (Y) at the position corresponding to position 9 of SEQ ID NO: 41, a lysine (K) at the position corresponding to position 13 of SEQ ID NO: 41, a tryptophan (W) at the position corresponding to position 18 of SEQ ID NO: 41, an arginine (R) at the position corresponding to position 47 of SEQ ID NO: 41; and a threonine (T) at the position corresponding to position 51 of SEQ ID NO: 41. More preferably, the hetero-dimeric binding protein has in the second (C-terminal) monomer a tyrosine (Y) at the position corresponding to position 10 of SEQ ID NO: 42, and a leucine (L) at the position corresponding to position 14 of SEQ ID NO: 42. Still more preferably, the hetero-dimeric binding protein has in the second (C-terminal) monomer a valine (V) at the position corresponding to position 17 of SEQ ID NO: 42, a glutamine (Q) at the position corresponding to position 28 of SEQ ID NO: 42, an alanine (A) at the position corresponding to position 32 of SEQ ID NO: 42, and a tryptophan (W) at the position corresponding to position 35 of SEQ ID NO: 42.

Still more preferred, the hetero-dimeric binding protein comprises in the first (N-terminal) monomer an isoleucine (I) at the position corresponding to position 42 of SEQ ID NO: 41, and an alanine (A) at the positions corresponding to positions 44 and 52 of SEQ ID NO: 41. Even more preferred, the hetero-dimeric binding protein may, additionally or alternatively, comprise in the second (C-terminal) monomer an isoleucine (I) at the position corresponding to position 44 of SEQ ID NO: 42, and an alanine (A) at the positions corresponding to positions 46 and 54 of SEQ ID NO: 42. In further preferred embodiments, the hetero-dimeric binding protein comprises in the second (C-terminal) monomer a lysine (K) at the position corresponding to position 49 of SEQ ID NO: 42. In various embodiments, the hetero-dimeric binding protein comprises a leucine (L) in the first (N-terminal) monomer at the position corresponding to position 40 of SEQ ID NO: 41, and in the second (C-terminal) monomer at the position corresponding to position 42 of SEQ ID NO: 42. In various embodiments, the amino acid at the position corresponding to position 52 of SEQ ID NO: 41 is a methionine (M).

The present invention encompasses a hetero-dimeric binding protein comprising a first (N-terminal) monomer and a second (C-terminal) monomer, wherein the binding protein has at least 90%, 91%, 92%, 93%, 94%, or 95%, sequence identity to SEQ ID NO: 43. Preferably, the binding protein has a binding affinity of less than 500 nM, more preferably less than 100 nM, for the spike protein or domains thereof. Preferably, the binding protein has a tyrosine (Y) at the position corresponding to position 9 of SEQ ID NO: 43, a lysine (K) at the position corresponding to position 13 of SEQ ID NO: 43, a tryptophan (W) at the position corresponding to position 18 of SEQ ID NO: 43, an arginine (R) at the position corresponding to position 47 of SEQ ID NO: 43; and a threonine (T) at the position corresponding to position 51 of SEQ ID NO: 43. More preferably, the hetero-dimeric binding protein has a tyrosine (Y) at the position corresponding to position 66 of SEQ ID NO: 43, and a leucine (L) at the position corresponding to position 70 of SEQ ID NO: 43. Still more preferably, the hetero-dimeric binding protein has a valine (V) at the position corresponding to position 73 of SEQ ID NO: 43, a glutamine (Q) at the position corresponding to position 84 of SEQ ID NO: 43, an alanine (A) at the position corresponding to position 88 of SEQ ID NO: 43, and a tryptophan (VO at the position corresponding to position 91 of SEQ ID NO: 43. Still more preferred, the hetero-dimeric binding protein comprises an isoleucine (I) at the position corresponding to position 42 of SEQ ID NO: 43, and an alanine (A) at the positions corresponding to positions 44 and 52 of SEQ ID NO: 43. Even more preferred, the hetero-dimeric binding protein may, additionally or alternatively, comprise an isoleucine (I) at the position corresponding to position 100 of SEQ ID NO: 43, and an alanine (A) at the positions corresponding to positions 102 and 110 of SEQ ID NO: 43. In further preferred embodiments, the hetero-dimeric binding protein comprises a lysine (K) at the position corresponding to position 105 of SEQ ID NO: 43. In various embodiments, the hetero-dimeric binding protein comprises a leucine (L) at the position corresponding to positions 40 and 98 of SEQ ID NO: 43. In various embodiments, the amino acid at the position corresponding to position 52 of SEQ ID NO: 43 is a methionine (M). Selected examples for dimeric S1/RBD binding proteins are provided in SEQ ID NOs: 7, 8, 17, 43-47, 66, and 67. A selected example for a trimer of a dimeric 51 and RBD binding protein is provided in SEQ ID NO: 47.

In some embodiments, the binding protein for the spike protein of SARS-Cov-2 is a multimer, preferably a dimer, that comprises an amino acid sequence with at least 90%, 91%, 92%, 93%, 94%, or 95%, sequence identity, to any one selected from the group of SEQ ID NOs: 3, 20, 21, preferably SEQ ID NO: 3, in N- or C-terminal position. Selected examples for dimeric S1/RBD binding proteins are provided in SEQ ID NOs: 9, 18, 34-36, and 57-62. For example, the hetero-multimeric (for example dimer) binding protein for the spike protein may comprise SEQ ID NO: 26 and SEQ ID NO: 3 linked to each other in head-to-tail orientation. In some embodiments, the binding protein for the spike protein or a domain thereof comprises SEQ ID NO: 3 and has at least 85% sequence identity to the amino sequence of SEQ ID NO: 9 (213103). For example, the hetero-multimeric binding protein for the spike protein comprises SEQ ID NO: 37 and SEQ ID NO: 3 linked to each other in head-to-tail orientation. In other embodiments, the binding protein for the spike protein or a domain thereof comprises SEQ ID NO: 3 and has at least 85% sequence identity to the amino sequence of SEQ ID NO: 36 (212880). For example, the hetero-multimeric (for example dimer) binding protein for the spike protein may comprise SEQ ID NO: 21 and SEQ ID NO: 22 linked to each other in head-to-tail orientation. In some embodiments, the binding protein for the spike protein or a domain thereof comprises SEQ ID NO: 21 and has at least 85% sequence identity to the amino sequence of SEQ ID NO: 18 (212728). For example, the hetero-multimeric (for example dimer) binding protein for the spike protein may comprise SEQ ID NO: 24 and SEQ ID NO: 25 linked to each other in head-to-tail orientation. In some embodiments, the binding protein for the spike protein or a domain thereof comprises SEQ ID NO: 24 and has at least 85% sequence identity to the amino sequence of SEQ ID NO: 19 (212522).

Additional moieties. In some embodiments, the binding protein for the spike protein or a domain thereof as described above further comprises or is fused to at least one further polypeptide distinct from the polypeptide as disclosed. In various embodiments, the further polypeptide fused to the binding protein for the spike protein and distinct from the binding protein for the spike protein as disclosed herein might be a non-Ig-binding protein, for example but not limited to, a protein that does not bind to the Fc part of immunoglobulin. In some embodiments, a non-Ig binding protein has at least 89.5% identity to SEQ ID NO: 13 or SEQ ID NO: 14. Accordingly, some embodiments encompass fusion proteins comprising a binding protein for the spike protein or domain thereof as disclosed herein and one or two or three or more non-Ig-binding polypeptide(s).

In some embodiments, a fusion protein may comprise the following combinations from N-terminus to C-terminus: (a) at least one binding protein for the spike protein fused to at least one non-Ig binding protein; (b) at least one non-Ig binding protein fused to at least one binding protein for the spike protein; (c) non-Ig binding protein fused to a binding protein for the spike protein fused to a binding non-Ig binding protein; (d) binding protein for the spike protein fused to a dimer of a non-Ig binding protein; (e) dimer of a binding protein for the spike protein fused to a dimer of a non-Ig binding protein; (f) dimer of a non-Ig binding protein fused to a binding protein for the spike protein; (g) non-Ig binding protein fused to a dimer of a binding protein for the spike protein fused to a non-Ig binding protein; (h) binding protein for the spike protein fused to a non-Ig binding protein fused to a binding protein for the spike protein fused to a non-Ig binding protein; (i) monomer or dimer of a binding protein for the spike protein fused to a dimer or tetramer of a non-Ig binding protein; (j) dimeric binding protein fused to a non-Ig binding protein (e.g. monomer, dimer or trimer) fused to a monomer of binding protein; (k) monomer of a binding protein fused to a non-Ig binding protein (e.g. monomer, dimer or trimer) fused to a dimeric binding protein. Other combinations of non-Ig binding protein and binding protein for the spike protein are also feasible to someone skilled in the art. A fusion of the binding protein with one, two, three or more non-Ig binding protein(s) may improve expression of the protein and ligand detectability in the Protein A ELISA leaching assay (see Examples).

As further described herein, the non-Ig-binding protein has no detectable binding affinity for the Fc domain of immunoglobulin as determined by SPR spectroscopy, more specifically the BIAcore SPR system. Further, the non-Ig binding protein has no detectable binding affinity for SARS Cov-2 and in particular no detectable binding affinity for the S1 domain or the RBD; as determined by SPR spectroscopy, more specifically the BIAcore SPR system.

In some embodiments, fusion proteins comprising at least one binding protein for the spike protein and at least one non-Ig binding protein are provided in SEQ ID NO: 29 (212895), SEQ ID NO: 30 (212896), SEQ ID NO: 31 (212897), SEQ ID NO: 32 (213147), SEQ ID NO: 33 (213152), SEQ ID NO: 38, SEQ ID NO: 39, SEQ ID NOs: 48-55, 68-72.

SEQ ID NO: 29 (212895) is a fusion protein wherein the spike protein binding protein of SEQ ID NO: 1 (212434) is fused to a trimer of non-Ig binding protein of SEQ ID NO: 14. SEQ ID NO: 31 (212897) is a fusion protein wherein the spike protein binding protein of SEQ ID NO: 1 (dimer of 212434) is fused to a tetramer of non-Ig binding protein of SEQ ID NO: 14. SEQ ID NO: 32 (213147) is a fusion protein wherein the spike protein binding protein of SEQ ID NO: 9 (dimer referred to as 213103) is fused to a dimer of non-Ig binding protein of SEQ ID NO: 14. SEQ ID NO: 33 (213152) is a fusion protein wherein the spike protein binding protein of SEQ ID NO: 18 (dimer referred to as 212728) is fused to a dimer of non-Ig binding protein of SEQ ID NO: 14.mSEQ ID NO: 38 is a fusion protein wherein the spike protein binding protein of SEQ ID NO: 28 is fused to a dimer non-Ig binding protein of SEQ ID NO: 14. SEQ ID NO: 30 (212896) is a fusion protein wherein the spike protein binding protein of SEQ ID NO: 28 is fused to a trimer of non-Ig binding protein of SEQ ID NO: 14. SEQ ID NO: 39 is a fusion protein wherein the spike protein binding protein of SEQ ID NO: 28 is fused to non-Ig binding protein of SEQ ID NO: 14 fused to spike protein binding protein of SEQ ID NO: 28 fused to non-Ig binding protein of SEQ ID NO: 14.

The present invention encompasses fusion proteins comprising a binding protein fused to a non-Ig binding protein of SEQ ID NO: 13 or SEQ ID NO: 14, wherein the binding protein is a dimer or multimer, and wherein in the N-terminal monomer of the dimer or multimer, the amino acids at the positions corresponding to positions 44, 46, and/or 54 in SEQ ID NO: 15 may not be V, G, and S, respectively. In various embodiments, the amino acids at the positions corresponding to positions 44, 46, and/or 54 in SEQ ID NO: 15 are I, A, and A, respectively. In various embodiments, the first monomer of the dimer or multimer may have at least 92%, at least 93%, or at least 94% sequence identity (i.e. 4 amino acids difference) to the sequence of SEQ ID NO: 2 or the sequence of SEQ ID NO: 40. In such embodiments, in the C-terminal monomer, the amino acid at the position corresponding to position 49 in SEQ ID NO: 15 may be K. In various embodiments, the second monomer of the dimer or multimer may have at least 93% sequence identity to the sequence of SEQ ID NO: 5.

The present invention encompasses fusion proteins comprising a binding protein fused to fused to a non-Ig binding protein of SEQ ID NO: 13 or SEQ ID NO: 14, wherein the binding protein is a dimer or multimer, and wherein the first (N-terminal) monomer of the dimer or multimer corresponds to the sequence of SEQ ID NO: 2, and wherein in the second (or C-terminal) monomer, the amino acids at the positions corresponding to positions 44, 46, and/or 54 in SEQ ID NO: 15 may not be V, G, and S, respectively. In various embodiments, in said second monomer, the amino acids at the positions corresponding to positions 44, 46, and/or 54 in SEQ ID NO: 15 may be I, A, and A, respectively. The second monomer of the dimer or multimer may have at least 90%, 91%, 92%, 93%, 94%, or 95%, sequence identity to the sequence of SEQ ID NO: 5. In such embodiments, in the second (or C-terminal) monomer, the amino acid at the position corresponding to position 49 in SEQ ID NO: 15 may be K. In various embodiments, the second monomer of the dimer or multimer may have at least 90%, 91%, 92%, 93%, 94%, or 95%, sequence identity to the sequence of SEQ ID NO: 5.

The present invention encompasses fusion proteins comprising multimeric binding proteins comprising a dimer according to SEQ ID NO: 43 and a monomer according to SEQ ID NO: 28 in head-to-tail orientation fused to at least one non-Ig binding protein, preferably according to SEQ ID NO: 14. The present invention also encompasses fusion proteins comprising multimeric binding proteins comprising a monomer according to SEQ ID NO: 28 and a dimer according to SEQ ID NO: 43 in head-to-tail orientation fused to at least one non-Ig binding protein, preferably according to SEQ ID NO: 14. SEQ ID NO: 51 is a fusion protein wherein the spike protein binding protein of SEQ ID NO: 43 is fused to at least one non-Ig binding protein, preferably according to SEQ ID NO: 14. Selected examples for fusion proteins comprising 51 and RBD binding proteins are shown in SEQ ID NOs: 29-33, 38, 39, 48-55, 68-72.

In some embodiments, the binding protein for the spike protein or a domain thereof as described above further comprises or is fused to or conjugated to a compound or at least one further polypeptide distinct from the polypeptide as disclosed. In various embodiments, the further compound or polypeptide distinct from the binding protein for the spike protein as disclosed herein might be at least one diagnostically active moiety. In some embodiments, the binding protein for the spike protein (or a domain thereof) is fused to or conjugated to a diagnostically active moiety optionally selected from a radionuclide, fluorescent protein, photosensitizer, dye, or enzyme, or any combination of the above. In some embodiments, a binding protein for the spike protein or a domain thereof that comprises additionally or is fused to or conjugated to at least one diagnostic moiety can be employed, for example, as imaging agent, for example to evaluate presence and distribution of viral particles. Methods for detection or monitoring of viruses may involve imaging methods. Such methods involve imaging SARS-Cov-2 or variants by, for example, radioimaging or photoluminescense or fluorescence. Suitable radionuclides for applications in imaging in vivo or in vitro or for radiotherapy include for example but are not limited to the group of gamma-emitting isotopes, the group of positron emitters, the group of beta-emitters, and the group of alpha-emitters. In some embodiments, suitable conjugation partners include chelators such as 1,4,7,10-tetraazacyclododecane-1,4,7,10-tetraacetic acid (DOTA) or diethylene triamine pentaacetic acid (DTPA) or their activated derivatives, nanoparticles and liposomes. In various embodiments, DOTA may be suitable as complexing agent for radioisotopes and other agents for imaging.

In some embodiments, the binding protein for the spike protein or a domain thereof as described above further comprises at least or is fused to or conjugated to a compound or one further polypeptide distinct from the polypeptide as disclosed. In various embodiments, the further polypeptide distinct from the binding protein for the spike protein as disclosed herein might be at least one therapeutically active moiety. In some embodiments, the binding protein for the spike protein (ora domain thereof) is fused to or conjugated to a therapeutically active moiety optionally selected from a monoclonal antibody or a fragment thereof, a radionuclide, a cytotoxic compound, a cytokine, a chemokine, an enzyme, or derivatives thereof, or any combination of the above. In some embodiments, the binding protein for the spike protein or a domain thereof as described above additionally comprises or is fused to or conjugated to a therapeutically active component and may be used in targeted delivery of any of the above listed components to the spike protein of SARS-Cov-2 or variants.

In some embodiments, the binding protein for the spike protein or a domain thereof as described above further comprises or is fused to or conjugated to a compound or at least one further polypeptide distinct from the polypeptide as disclosed. In various embodiments, the compound or further polypeptide distinct from the binding protein for the spike protein as disclosed herein might be at least one moiety modulating pharmacokinetics. In some embodiments, the binding protein for the spike protein (or a domain thereof) is fused to or conjugated to a moiety modulating pharmacokinetics optionally selected from a polyethylene glycol, a human serum albumin, an albumin-binding protein or peptide, an immunoglobulin binding protein or peptide, or an immunoglobulin or immunoglobulin fragment, a polysaccharide, or an unstructured amino acid sequence comprising amino acids alanine, glycine, serine, proline.

The moieties, i.e. the binding protein for the spike protein and the moiety modulating pharmacokinetics or diagnostically active moiety or therapeutically active moiety, may be linked to each other directly head-to-tail or may be linked by a linker, wherein the linker preferably is a peptide linker. In various embodiments, a peptide linker may be considered as an amino acid sequence which sterically separates the two portions of the fusion protein. Typically, such linker consists of between 1 and 30 amino acids.

In some embodiments, the binding protein for the spike protein may comprise at least two monomers SEQ ID NO: 1 (or at least 80% identical thereto) linked to each other in head-to-tail orientation via a peptide linker. For example, the binding protein for the spike protein may comprise at least two monomers as shown in SEQ ID NO: 28 (or at least 80% identical thereto) linked to each other in head-to-tail orientation via a peptide linker between two monomers. In some embodiments, the linker may consist of 20 amino acids selected from any amino acid. In some embodiments, the linker may consist of 20 amino acids selected from the group of Q, A, P, K, V, D, F, S.

Molecules for purification or detection. In some embodiments, the binding protein for the spike protein or domains thereof may also comprise additional amino acid residues at the N- and/or C-terminal end, such as for example an additional sequence at the N- and/or C-terminal end. Additional sequences may include for example sequences introduced e.g. for purification or detection. Typical examples for such sequences include, without being limiting, Strep-tags, oligohistidine-tags, glutathione S-transferase, maltose-binding protein, inteins, intein fragments, or the albumin-binding domain of protein G, or others. In one embodiment, additional amino acid sequences include one or more peptide sequences that confer an affinity to certain chromatography column materials. The binding protein for the spike protein or domains thereof may include specific attachment sites for the attachment to solid supports, preferably at the C-terminal end, such as cysteine or lysine.

Method of identification/generation of the binding protein for the spike protein. The present invention further provides a method for the identification/generation of a binding polypeptide for the spike glycoprotein or domains thereof as disclosed herein with binding affinity for the spike protein or domains thereof, the method comprising the following steps: (i) providing a population (library) of proteins; (ii) contacting the population of proteins of (i) with a protein comprising spike protein or a domain thereof; (iii) identifying a complex comprising an binding protein for the spike protein or domain thereof bound to protein comprising spike protein or a domain thereof; and (iv) obtaining an binding protein for the spike protein or domain thereof is capable of binding to protein comprising spike protein or a domain thereof.

The method for the generation of a binding protein for the spike protein or domain thereof may comprise, a further step of determining the binding affinity to a protein comprising spike protein or a domain thereof. The binding affinity may be determined as described elsewhere herein.

Use of the novel binding protein for the spike protein in technical applications. Also provided herein is the use of any novel binding protein for the spike protein as disclosed herein, including multimers, including fusion proteins, in technical applications, preferably for use in affinity purification. This is particularly important in view of purification of vaccines against SARS-Cov-2 or variants.

As described herein, affinity chromatography (also called affinity purification) makes use of specific binding interactions between molecules. Methods for immobilization of protein and methods for affinity chromatography are well-known in the field of protein purification and can be easily performed by a skilled person in this field using standard techniques and equipment. Further embodiments relate to a process of manufacturing spike protein or domains thereof or a vaccine comprising spike protein or domains thereof comprising at least one chromatographic step employing an affinity chromatography matrix having an affinity for specifically binding spike protein or domains thereof wherein the affinity ligand (binding protein) for spike protein or domains thereof as described above is coupled to said affinity chromatography matrix.

In various embodiments, the method of affinity purification may further comprise one or more washing steps carried out under conditions sufficient to remove from the affinity purification matrix some or all molecules that are non-specifically bound thereto. Affinity purification matrices suitable for the disclosed uses and methods are known to a person skilled in the art.

Conjugation to a solid support. In various aspects and/or embodiments of the present invention, the novel proteins disclosed herein including novel proteins generated or obtained by any of the methods as described above are conjugated to a solid support. In some embodiments of the invention, the polypeptide comprises an attachment site for site-specific covalent coupling of the polypeptide to a solid support. Specific attachment sites comprise without being limited thereto, natural amino acids, such as cysteine or lysine, which enable specific chemical reactions with a reactive group of the solid phase, or a linker between the solid phase and the protein.

Affinity purification matrix. In another embodiment, an affinity purification matrix is provided comprising a binding protein for the spike protein, including a polypeptide identified by any of the methods as described above.

In preferred embodiments, the affinity purification matrix is a solid support. The affinity purification matrix comprises at least one binding protein for the spike protein or domains thereof as described herein. Accordingly, a novel binding protein for the spike protein or domains thereof disclosed herein is encompassed for use in the purification of a protein or particle by an affinity matrix, preferably for use in the purification of a vaccine against SARS-Cov-2 or variants thereof.

Solid support matrices for affinity chromatography are known in the art and include, e.g., without being limited thereto, agarose and stabilized derivatives of agarose, cellulose or derivatives of cellulose, controlled pore glass, monolith, silica, zirconium oxide, titanium oxide, or synthetic polymers, and hydrogels of various compositions and combinations of the above.

The formats for solid support matrices can be of any suitable well-known kind. Such solid support matrix for coupling a novel protein or polypeptide of the present invention might comprise, e.g., one of the following, without being limited thereto: columns, capillaries, particles, membranes, filters, monoliths, fibers, pads, gels, slides, plates, cassettes, or any other format commonly used in chromatography and known to someone skilled in the art.

In one embodiment, the matrix is comprised of substantially spherical beads, for example Sepharose or Agarose beads. Matrices in particle form can be used as a packed bed or in a suspended form including expanded beds. In other embodiments of the invention, the solid support matrix is a membrane, for example a hydrogel membrane. In some embodiments, the affinity purification may involve a membrane as a matrix to which a binding protein for the spike protein of the present invention is covalently bound. The solid support can also be in the form of a membrane in a cartridge.

In some embodiments, the affinity purification involves a chromatography column containing a solid support matrix to which a novel protein of the present invention is covalently bound. A novel protein or polypeptide of the present invention may be attached to a suitable solid support matrix via conventional coupling techniques. Methods for immobilization of protein ligands to solid supports are well-known in the field of protein engineering and purification and can easily be performed by a skilled person in this field using standard techniques and equipment.

Further, in some embodiments, the binding protein for the spike protein as described herein or the fusion protein as described herein are used in methods to determine the presence of a protein comprising spike protein or a domain thereof, for example, a vaccine against SARS-Cov-2. Some embodiments relate to a method of analyzing the presence of protein comprising spike protein or a domain thereof in liquid samples, the method comprising the following steps: (a) providing a liquid that contains a protein or particle comprising spike protein or a domain thereof, (b) providing the binding protein for the spike protein, (c) contacting the liquid that contains protein or particle comprising spike protein or a domain thereof with the binding protein for the spike protein as described herein under conditions that permit binding of the at least one binding protein for the spike protein to protein or particle comprising spike protein or a domain thereof, (d) isolating (eluting) the complex of a protein or particle comprising spike protein or a domain thereof and the binding protein for the spike protein, and optionally, (e) determining the amount of the binding protein for the spike protein which indicates the amount of protein or particle comprising spike protein or a domain thereof in the liquid of (a).

Further embodiments relate to a method of quantification of a protein or particle comprising spike protein or a domain thereof of the spike protein, the method comprising: (a) providing a liquid that contains protein or particle comprising spike protein or a domain thereof; (b) providing a matrix to which the binding protein for the spike protein as described herein has been covalently coupled; (c) contacting said affinity purification matrix with the liquid under conditions that permit binding of the at least one binding protein for the spike protein to protein or particle comprising spike protein or a domain thereof; (d) eluting said protein or particle comprising spike protein or a domain thereof; and optionally, (e) quantitating the amount of eluted protein or particle comprising spike protein or a domain thereof. Methods to determine the presence of protein or particle comprising spike protein or a domain thereof in liquid samples might be quantitative or qualitative. Such methods are well known to the skilled person and can be selected, for instance but limited to, from the following methods that are well established in the art: enzyme-linked immunosorbent assay (ELISA), enzymatic reactions, surface plasmon resonance (SPR) or chromatography.

Use of the binding proteins for spike protein for medical applications. In some embodiments, the binding protein for the spike protein or a domain thereof as described above is used in diagnosis or treatment of SARS-COV-2 (or variants thereof) related diseases. In one embodiment, the binding protein for the spike protein or domains thereof is used in medicine to diagnose of SARS-Cov-2 or variants or treat diseases associated with SARS-Cov-2 or variants. One embodiment is a method of diagnosing (including monitoring), the method of diagnosis (monitoring) comprising administering to the subject the binding protein for the spike protein or domains thereof as described, optionally conjugated to radioactive molecules. In various embodiments, the binding protein for the spike protein or domains thereof as disclosed herein may be used for diagnosis of SARS-Cov-2 or variants, optionally wherein the binding protein for the spike protein or domains thereof is conjugated to a radioactive molecule. In some embodiments, imaging methods using the binding protein for the spike protein or domains thereof with labels such as radioactive or fluorescent can be employed to visualize spike protein or domains thereof on virus cells, for example, to evaluate presence of viral particles, and/or to evaluate the response of a patient to a therapeutic treatment. In some embodiments, the diagnostic methods are in vitro methods for diagnosing the presence of viral particles by using the binding protein for the spike protein (i.e. binding protein for S1 domain and/or RBD and/or variants thereof) to evaluate the presence of viral particles. Some embodiments relate to the use of the binding protein for the spike protein (i.e. binding protein for S1 domain and/or RBD) for the manufacture of a substance or compound for the diagnosis of diseases related to or caused by SARS-Cov-2 or variants. One embodiment is a method of treating a subject having diseases related to or caused by SARS-Cov-2 or variants, the method of treatment comprising administering to the subject the spike protein binding protein as described herein. In various embodiments, the binding protein for the spike protein or domains thereof as disclosed herein is for use for treatment of diseases related to or caused by SARS-Cov-2 or variants. Some embodiments relate to the use of the binding protein for the spike protein (i.e. binding protein for S1 domain and/or RBD) for the manufacture of a medicament for the treatment of diseases related to or caused by SARS-Cov-2 or variants.

Compositions. Various embodiments relate to a composition comprising the binding protein for the spike protein or domains thereof as disclosed herein. A composition comprising the binding protein for the spike protein or domains thereof as defined above for use in medicine, preferably for use in the diagnosis (detection/monitoring) of SARS-Cov-2 or treatment of diseases related to or caused by SARS-Cov-2 or variants as described above. Compositions comprising the binding protein for the spike protein or domains thereof as described above may be used for clinical applications for both diagnostic and therapeutic purposes. In particular, compositions comprising the binding protein for the spike protein or domains thereof as described herein may be used for clinical applications for imaging, monitoring, and eliminating or inactivating SARS-Cov-2 or variant.

Various embodiments relate to a diagnostic composition for the diagnosis of SARS-Cov-2 comprising the binding protein for the spike protein or domains thereof as defined herein and a diagnostically acceptable carrier and/or diluent. These include for example but are not limited to stabilizing agents, surface-active agents, salts, buffers, coloring agents etc. The compositions can be in the form of a liquid preparation, a lyophilisate, granules, in the form of an emulsion or a liposomal preparation.

The diagnostic composition comprising the binding protein for the spike protein or domains thereof as described herein can be used for diagnosis of SARS-Cov-2 or variants, as described above. Various embodiments relate to a pharmaceutical (e.g. therapeutic) composition for the treatment of diseases comprising the binding protein for the spike protein or domains thereof as disclosed herein, and a pharmaceutically (e.g. therapeutically) acceptable carrier and/or diluent. The pharmaceutical (e.g. therapeutic) composition optionally may contain further auxiliary agents and excipients known per se. These include for example but are not limited to stabilizing agents, surface-active agents, salts, buffers, coloring agents etc.

The pharmaceutical composition comprising the binding protein for the spike protein or domains thereof as defined herein can be used for treatment of diseases, as described above.

The compositions contain an effective dose of the binding protein for the spike protein or domains thereof as defined herein. The amount of protein to be administered depends on the organism, the type of disease, the age and weight of the patient and further factors known perse. Depending on the galenic preparation these compositions can be administered parentally by injection or infusion, systemically, intraperitoneally, intramuscularly, subcutaneously, transdermally, or by other conventionally employed methods of application. The composition can be in the form of a liquid preparation, a lyophilisate, a cream, a lotion for topical application, an aerosol, in the form of powders, granules, in the form of an emulsion or a liposomal preparation. The type of preparation depends on the type of disease, the route of administration, the severity of the disease, the patient and other factors known to those skilled in the art of medicine.

The various components of the composition may be packaged as a kit with instructions for use.

Polynucleotides, vectors, host cells. One embodiment covers an isolated polynucleotide or nucleic acid molecule encoding a binding protein for the spike protein as disclosed herein. A further embodiment also encompasses proteins encoded by the polynucleotides as disclosed herein.

Further provided is a vector, in particular an expression vector, comprising the isolated polynucleotide or nucleic acid molecule of the invention, as well as a host cell comprising the isolated polynucleotide or the expression vector. For example, one or more polynucleotides, which encode a polypeptide as disclosed herein may be expressed in a suitable host and the produced protein can be isolated. A vector means any molecule or entity (e.g., nucleic acid, plasmid, bacteriophage or virus) that can be used for transfer of protein-encoding information into a host cell. Suitable vectors that may be applied in the present invention are known in the art. Furthermore, an isolated cell comprising a polynucleotide or nucleic acid, or a vector as disclosed herein is provided. Suitable host cells include prokaryotes or eukaryotes, for example a bacterial host cell, a yeast host cell or a non-human host cell carrying a vector. Suitable bacterial expression host cells or systems are known in the art. Various mammalian or insect cell culture systems as known in the art can also be employed to express recombinant proteins.

Method of producing a protein of the invention. In a further embodiment, a method for the production of the binding protein for the spike protein or domain thereof as described is provided, the method comprising the step(s): (a) culturing a (suitable) host cell under conditions suitable for the expression of the binding protein for the spike protein or domain thereof so as to obtain said binding protein for the spike protein; and (b) optionally isolating said binding protein for the spike protein or domain thereof. Suitable conditions for culturing a prokaryotic or eukaryotic host are well known to a person skilled in the art.

The binding protein for the spike protein or domain thereof may be prepared by any conventional and well-known techniques such as plain organic synthetic strategies, solid phase-assisted synthesis techniques, or by commercially available automated synthesizers. They may also be prepared by conventional recombinant techniques, alone or in combination with conventional synthetic techniques. In one embodiment, a method for the preparation of the binding protein for the spike protein or domain thereof is provided, as detailed above, said method comprising the steps: (a) providing a nucleic acid molecule encoding the binding polypeptide; (b) introducing said nucleic acid molecule into an expression vector; (c) introducing said expression vector into a host cell; (d) culturing the host cell in a culture medium; (e) subjecting the host cell to culturing conditions suitable for expression thereby producing a binding polypeptide; optionally (f) isolating the protein or polypeptide produced in step (e); and (g) optionally conjugating the protein or polypeptide to a solid matrix as described above. In various embodiments of the present invention the production of the binding protein for the spike protein is performed by cell-free in vitro transcription and translation.

The following Examples are provided for further illustration of the invention. The invention, however, is not limited thereto, and the following Examples merely show the practicability of the invention on the basis of the above description.

### EXAMPLES

The following Examples are provided for further illustration of the invention. The invention, however, is not limited thereto, and the following Examples merely show the practicability of the invention on the basis of the above description. For a complete disclosure of the invention reference is made also to the literature cited in the application which is incorporated completely into the application by reference.

### Example 1. Selection and Screening of Binding Protein for the Spike Protein

Validation of the target: The binding of the S1 or RBD subunit of the spike protein to ACE2 was analyzed via SPR (see Example 3 below). Chip: Immobilized ACE2-Avi-His (biotinylated) on SA Chip (−1000 RU); analyte concentration: 100 nM. Both the S1 subunit (S1-His) of the spike protein as well as the receptor binding domain (RBD) of the spike protein bind to immobilized ACE2-receptor.

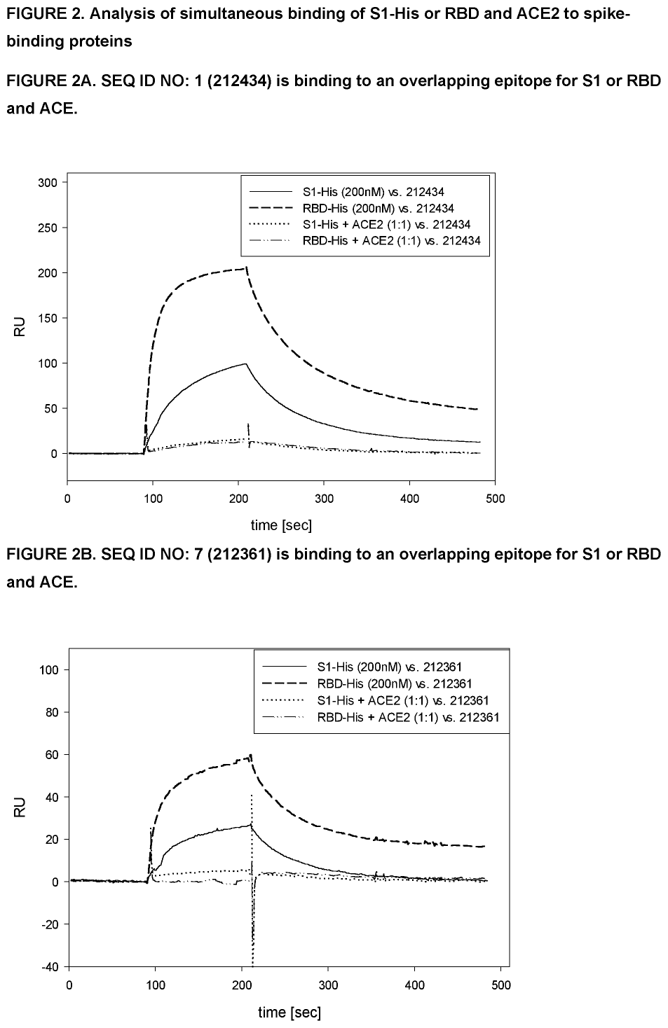

Proprietary cDNA libraries based on stable Protein A like variants (artificial mosaic proteins composed of fragments of Protein A domains and additional mutations; e.g. SEQ ID NO: 15 or SEQ ID NO: 16) were synthesized in house by randomized oligonucleotides generated by synthetic trinucleotide phosphoramidites (ELLA Biotech) to achieve a well-balanced amino acid distribution with simultaneously exclusion of cysteine and other amino acid residues at randomized positions. For the following selection process by ribosome display these cDNA libraries were supplemented with a T7 promoter region at the 5′ end and a spacer region at the 3′ end, respectively. Unless otherwise indicated, established recombinant genetic methods were used. Proprietary cDNA libraries based on stable Protein A derived proteins including the ribosome display regulatory elements were each transcribed into the corresponding RNA library followed by in vitro translation into a protein library. Those generated mRNA-ribosome-protein-ternary complexes were stable and thus suitable for selection. The ternary complexes were allowed to bind the target protein already immobilized on magnetic epoxy beads (Dynabeads™ M-270 Epoxy, Thermo Fisher Scientific). Target concentration started at 120 nM (round 1) and declined each round down to 10 nM (round 4). Selected pools of round 3 and 4 were amplified by PCR according to methods known in the art, cut with appropriate restriction nucleases and ligated into a derivative of the expression vector pET-28a (Merck, Germany) comprising an N-terminal GFP-His-tag followed by an enzymatic cleavage site and a C-terminal cysteine. To identify SARS-Cov2 specific pools, pools of round 3 and 4 were analyzed by pool ELISA. Therefore, subcloned pools were transformed in E. coli BL21(DE3), cultivated in 3 ml autoinduction media and the cells were harvested by three freeze/thaw cycles. Wells of a high binding black plate (Greiner Bio-One) were coated with S1-His protein and RBD-Fc (each 2.5 μg/ml), respectively. E. coli lysates were allowed to bind the targets followed by several washing steps. Bound variants were detected via fluorescence signal (ex 485 nm/em 535 nm). Results: 3 selection pools from proprietary libraries based on stable Protein A like variants (e.g. cs27) showed specific binding to the unmodified SARS-Cov2 S1-His protein and/or RBD-Fc. Primary screening—Selection pools were therefore proceeded to high throughput primary screening. Positive control: sfGFP-10×His-cs5 (Ig binding protein), negative control: sfGFP-10×His. Detection: fluorescence signal (ex 485 nm/em 535 nm). 5671 variants were selected for secondary screening (on target: S1-His (c=2.5 μg/ml); off target: BSA). Hit criteria: signal of sample larger than signal of negative control. 177 hits were identified for further analysis. Secondary screening. 177 hits were sequenced, produced in m-scale (Phynexus) (see Example 2) and analyzed by BLI. The proteins were immobilized on a Ni-NTA sensor (ForteBio). Upon binding, target analyte was accumulated on the surface increasing the refractive index. This change in the refractive index was measured in real time and plotted as nm shift versus time (see FIG. 2B).

### Example 2. Expression and Purification of SARS-Cov2 Binding Proteins

Variants with SARS Cov2 S1 binding were expressed in Escherichia coli BL21(DE3) using a pNP-013 vector system under regulation of a T7 promoter. Proteins were produced in soluble form after induction by lactose included in the medium (autoinduction medium). BL21 (DE3) competent cells were transformed with the expression plasmid, spread onto selective agar plates (kanamycin) and incubated over night at 37° C. Precultures were inoculated from single colony in 3 ml 2×YT medium supplemented with 50 μg/ml kanamycin and cultured for 6 h at 37° C. at 210 rpm in a conventional orbital shaker in culture tubes. For main cultures 350 mL ZYM-5052 medium (see Studier 2004) were inoculated with entire precultures and incubated in 2.5 L Ultra Yield™ flasks at 30° C. at 180 rpm in an orbital shaker. The culture medium was supplemented with 50 μg/ml kanamycin and antifoam SE15. Recombinant protein expression was induced by metabolizing glucose and subsequently allowing lactose to enter the cells. Cells were grown over night for approximately 18 hours to reach a final OD600 of about 10-20. Before the harvest, the OD600 was measured, samples adjusted to 0.6/OD600 were withdrawn, pelleted and frozen at −20° C. To collect biomass cells were centrifuged at 12000×g for 20 min at 22° C. Pellets were weighed (wet weight) and stored at −20° C. before processing.

Fusion proteins were expressed by E. coli BL21(DE3) through fermentation using a tag-free proprietary pET-based vector system (e.g. referred to as pNP-004, featuring a c-terminal cysteine, or referred to as pNP-013, featuring an N-terminal superfolder-GFP-10×His-TVMV and C-terminal cysteine).

Precultures were generated in two steps. For a first preculture, 50 mL preculture medium (34.5 g/L yeast extract, 0.61 g/L MgSO₄, 14.2 K₂HPO₄, 0.5 g/L NH 4 CI, 20 g/L glucose, 50 μg/mL kanamycin) was inoculated from a single colony and incubated for 16-18 h at 37° C. and 210 rpm in a shake flask. The seed culture (second preculture) was inoculated to an OD600 of 0.1 and grown for 6 h at 37° C. and 210 rpm in 1 L baffled shake flasks with 200 mLs of preculture medium. The fermentation process was performed in a bench-top bioreactor as a fed-batch process. In the batch phase (37° C., pH 7.1, 30% pO₂ saturation, aeration 2 VVM), the culture medium (17.25 g/L yeast extract, 0.61 g/L MgSO₄, 14.2 K₂HPO₄, 0.5 g/L NH 4 CI, 50 μg/mL kanamycin) was inoculated to an OD600 of 0.3 and the culture was incubated until substrate was depleted (pO₂ spike). The pH value was maintained by automatic addition of potassium hydroxide (20% (w/v)) and phosphoric acid (10% (v/v)). Afterwards, the feeding phase was performed for 11 h (exponential growth, p=0.11 1/h) with glucose as the main substrate (200 g/L glucose, 276 g/L yeast extract, 50 μg/mL kanamycin). Protein expression was induced by isopropyl β-D-1-thiogalactopyranoside (IPTG, end concentration of 1 mM) at 30° C. for 5 h at a constant feeding rate (value as after 11 h of exponential feeding). Harvest was performed as described in the previous process.

Proteins were purified by affinity chromatography, protease cleavage and size exclusion. The initial capturing step was performed using IMAC (HisTrap HP 5 ml, GE Healthcare, binding buffer: 50 mM NaH2PO4, 500 mM NaCl, 30 mM Imidazole, 1 mM DTT pH 7.2; elution buffer: 50 mM NaH2PO4, 500 mM NaCl, 30 mM Imidazole, 1 mM DTT pH 7.2) followed by a desalting (HiPrep Desalting 53 ml, GE Healthcare) in 50 mM NaH₂PO₄, 150 mM NaCl, 10 mM Imidazole, 1 mM DTT pH 7.2 using an ÄKTA xpress system. The collected fractions with protein of interest were incubated with TVMV protease (1 μg TVMV to 100 μg protein) over night at room temperature. The proteins were further purified using IMAC (HisTrap HP 5 ml, GE Healthcare, binding buffer: 50 mM NaH₂PO₄, 500 mM NaCl, 10 mM Imidazole, 1 mM DTT pH 7.2; elution buffer: 50 mM NaH₂PO₄, 500 mM NaCl, 500 mM Imidazole, 1 mM DTT pH 7.2) in flow-through mode. TVMV protease and cleavage products were captured by the IMAC using an ÄKTA xpress system. The flow-through was collected and polished by size exclusion chromatography using a Superdex 75 26/600 column (GE Healthcare; buffer: 20 mM citric acid, 150 mM NaCl, 1 mM EDTA pH 6) carried out on an ÄKTA avant system (GE Healthcare). Variants without affinity tag (212895, 212896 and 212897) were captured via Q-Sepharose (GE Healthcare, binding buffer: 20 mM TRIS, 1 mM DTT, 1 mM EDTA pH 8.5; elution buffer: 20 mM TRIS, 1 mM DTT, 1 mM EDTA, 1M NaCl, pH 8.5) followed by Phenyl-HP-Sepharose (GE Healthcare, binding buffer: 20 mM TRIS, 1 mM EDTA, 1 mM DTT, 1M (NH₄)₂SO₄, pH 8.5; elution buffer: 20 mM TRIS, 1 mM EDTA, 1 mM DTT, pH 8.5) Fusion proteins were purified by hlgG-Sepharose according to manufactures instructions. Polishing was performed by size exclusion chromatography using a Superdex 75 or 20026/600 column (GE Healthcare; buffer: 20 mM citric acid, 150 mM NaCl, 1 mM EDTA pH 6) carried out on an ÄKTA avant system (GE Healthcare). Following SDS-PAGE analysis positive fractions were pooled and the protein concentrations were determined by absorbance measurement at 280 nm using the molar absorbent coefficient. Further analysis included RP-HPLC and SE-HPLC. Reversed phase chromatography (RP-HPLC) has been performed using a Dionex HPLC system and a PLRP-S(5 pm, 300 A) column (Agilent). Analytic size exclusion chromatography (SE-HPLC) has been performed using a Dionex HPLC system and a Superdex75 increase 5/150 GL (GE Healthcare).

TABLE 1A
Analytic results of spike protein binding proteins

| SEQ | | | | | | Solubility |
|---|---|---|---|---|---|---|
| ID | | SE-HPLC | rp-HPLC | YieldPerVolume | YieldPerWeight | Soluble |
| NO: | CID | Monomer | Hauptpeak | Yield per L | Yield per g | Expression |
| 9 | 213103 | 100% | 82.2% | 7.54 mg/L | 0.32 mg/g | 60% |
| 18 | 212728 | 100% | 61.3% | 3.66 mg/L | 0.18 mg/g | 50% |
| 19 | 212522 | 100% | 91.5% | 9.59 mg/L | 0.47 mg/g | 90% |
| 1 | 212434 | 100% | 97.8% | 49.29 mg/L | 1.29 mg/g | 80% |
| 7 | 212361 | 100% | 82.0% | 4.76 mg/L | 0.24 mg/g | 70% |
| 8 | 212335 | 100% | 81.0% | 1.54 mg/L | 80 mg/g | 60% |
| 17 | 212860 | 100% | 83.9% | 4.56 mg/L | 0.19 mg/g | 60% |

TABLE 1B
Analytic results of spike protein binding proteins

| SEQ ID | | SE-HPLC | rp-HPLC | Solubility |
|---|---|---|---|---|
| NO: | CID | Monomer | Hauptpeak | Soluble Expression |
| 29 | 212895 | 94.8% | 97.2% | 100% |
| 30 | 212896 | 86.6% | 65.5% | 100% |
| 31 | 212897 | 100% | 93.7% | 100% |
| 32 | 213147 | 100% | 91.5% | 100% |
| 33 | 213152 | 91.3% | 100% | 60% |
| 47 | 217797 | 97.2% | 95.9% | 90% |
| 49 | 217798 | 99.4% | 96.5% | 90% |
| 50 | 217799 | 98.6% | 90.8% | 60% |
| 51 | 217800 | 100% | 94.6% | 95% |

### Example 3. Binding Analysis of Proteins by SPR

The purified proteins were immobilized on a CM-5 sensor chip (GE Healthcare) using PDEA after NHS/EDC activation resulting in 110-140 RU with a Biacore 3000 system (GE Healthcare). The chip was equilibrated with SPR running buffer (PBS 0.05% Tween pH 7.3). Upon binding, target analyte was accumulated on the surface increasing the refractive index.

This change in the refractive index was measured in real time and plotted as response or resonance units versus time. The analytes (for example, SARS-CoV2 S protein or SARS-CoV2 S1 protein or SARS-CoV2 RBD, or SARS-CoV-1 or MERS-Cov or mutant proteins) were applied to the chip in serial dilutions with a flow rate of 30 ml/min. The association was performed for 120 seconds and the dissociation for 120 seconds. After each run, the chip surface was regenerated with 30 ml regeneration buffer (10 mM glycine pH 2.0) and equilibrated with running buffer. Binding studies were carried out by the use of the BIAcore 3000 (GE Healthcare); data evaluation was operated via the BIAevaluation 3.0 software, provided by the manufacturer, by the use of the Langmuir 1:1 model (RI=0). Evaluated dissociation constants (K D) were standardized against the immobilized protein and indicated. Shown is the change in refractive index measured in real time and plotted as response or resonance unit [RU] versus time [sec]. Results are shown in Table 2. SEQ ID NO: 15 (cs27), SEQ ID NO: 16 (C27), and the non-Ig binding protein (PAdelFc) do not bind to S1 or RBD and do not bind to variants of S1 or RBD.

TABLE 2
Binding affinity of binding proteins for spike protein and of
fusion proteins comprising spike protein binding proteins

| | | SPR | | SPR |
|---|---|---|---|---|
| | CID | KD_S1 | | KD_RBD |
| SEQ ID NO: | | | | |
| 1 | 212434 | 3.8 | nM | 10.2 nM |
| 7 | 212361 | 31 | nM | 21.3 nM |
| 8 | 212335 | 13 | nM | 68.8 nM |
| 17 | 212860 | 15 | nM | n.d. |
| 9 | 213103 | 8 | nM | n.d. |
| 18 | 212728 | 6.5 | nM | n.d. |
| 36 | 212880 | 9.1 | nM | n.d. |
| 19 | 212522 | 24.1 | nM | 27.1 nM |
| Fusion Proteins | | | | |
| 29 | 212895 | 560 | nM | 34.7 nM |
| 30 | 212896 | 113 | nM | 27.6 nM |
| 31 | 212897 | 233 | nM | 39.4 nM |
| 32 | 213147 | 225 | nM | 42.9 nM |
| 33 | 213152 | 112 | nM | 29.3 nM |

Ligands 212361, 212335, 212728, and fusion protein of SEQ ID NO: 32 (213147) showed binding to mutant of SARS CoV-2 S protein (69del, 70del, Y144del, N501Y, A570D, D614G, P681H, T716I, S982A, D1118H) (corresponds to B.1.1.7). Ligands 212361 and 212335 as well as the trimer of 212361 (217797) and fusion proteins 217798 and 217800 showed binding to SARS-CoV-2 S protein RBD (K417N, E484K, N501Y) or to SARS-CoV-2 S protein (L18F, D80A, D215G, R2451, K417N, E484K, N501Y, D614G, A701V) (corresponds to B.1.351). Fusion proteins 217800 and 217798 and the trimer of 212361 (217797) bind to SARS CoV-2 S protein. Fusion protein 217799 showed binding to SARS CoV-2 protein RBD.

### Example 4. Affinity Purification of a Protein Comprising Spike Protein or a Domain Thereof

TABLE 3
Dimers and fusion proteins comprising binding proteins for spike protein

| monomer 1 | monomer 2 | dimer | dimer | |
|---|---|---|---|---|
| SEQ ID NO: | SEQ ID NO: | SEQ ID NO: | CID | Comprised in fusion protein |
| 2 | 23 | 17 | 212860 | |
| 26 | 3 | 9 | 213103 | 213147 (SEQ ID NO: 32) |
| 33 | 3 | 36 | 212880 | |
| 21 | 22 | 18 | 212728 | 213152 (SEQ ID NO: 33) |
| 24 | 25 | 19 | 212522 | |
| 1 | 1 | | | 212897 (SEQ ID NO: 31) |
| 28 | 28 | | | 212896 (SEQ ID NO: 30) |
| 2 | 5 | 7 | 212361 | 217238 (SEQ ID NO: 48) |
| 2 | 42 | 44 | | 217798 (SEQ ID NO: 49); |
| | | | | 217799 (SEQ ID NO: 50) |
| 41 | 42 | 43 | | 217800 (SEQ ID NO: 51) |

Coupling parameter. Fusion proteins were purified to homogeneity and coupled for AIC experiments. Inhouse expressed target from Expi293 cells was used as target. Purified fusion proteins comprising the binding protein for the spike protein (e.g. 212895, 212896, 212897, 213147, 213152, 217797, 217798, 217799, 217800) were immobilized at 20-30 mg per mL activated Praesto™ Epoxy 85 (Purolite) according to the manufacturer's instructions, coupling conditions: 35° C. for 3 h, pH 9.5, 110 mg Na₂SO₄ per mL Resin. All ligands were successfully coupled to epoxy-activated Praesto 85 resin.

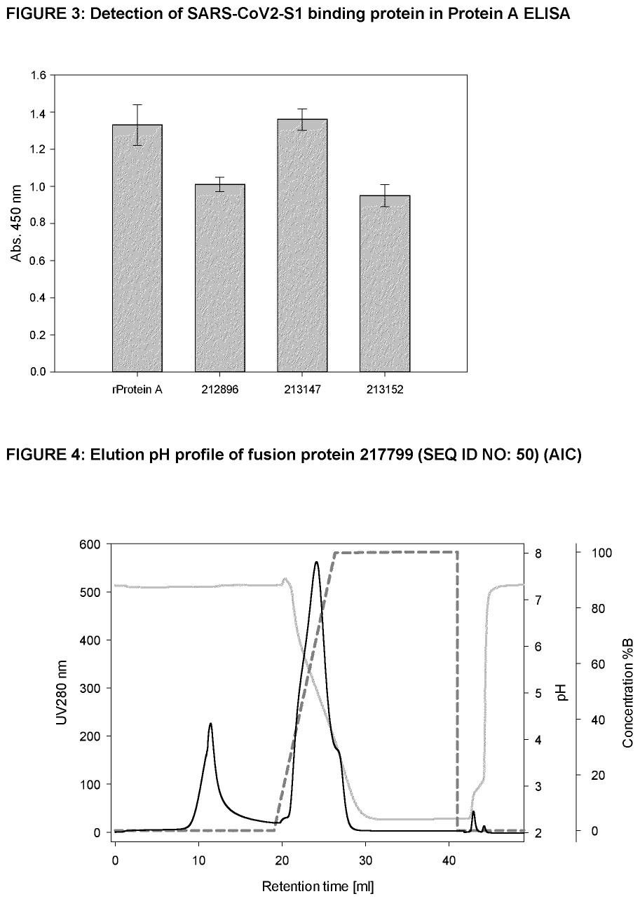

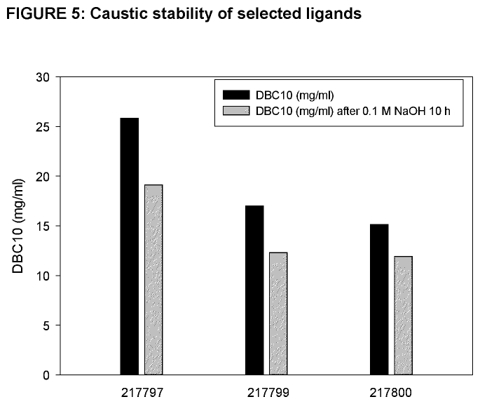

AIC experiments (Elution profile). For elution pH determination, resins were packed into superformance column housing (Götec, 5-50) and equilibrated in 1×PBS, pH 7.3. Resin was loaded with 1 mg of SARS-Cov2-RBD-His expressed in Expi293-F cell culture supernatant. Elution was performed with a gradient from pH 6.0 to pH 2.0 in 15 column volumes (CV) using a 100 mM citric acid buffer. The pH of buffer fractions containing the target was determined. The pH of the main fraction is listed in TABLE 4. FIG. 4 shows the elution pH profile of fusion protein 217799 (comprising SEQ ID NO: 2).

TABLE 4
Determined elution pH at main fraction

| SEQ ID NO: | CID | Elution pH |
|---|---|---|
| 7 | 212361 | >6.0 |
| 8 | 212335 | >6.0 |
| 29 | 212895 | 3.5 |
| 30 | 212896 | 3.4 |
| 31 | 212897 | 3.4 |
| 32 | 213147 | 4.6 |
| 33 | 213152 | 4.2 |
| 49 | 217798 | 5.0 |
| 50 | 217799 | 5.0 |
| 51 | 217800 | 4.6 |
| 47 | 217797 | 4.7 |

All fusion proteins comprising binding protein SEQ ID NO: 1 (212895, 212897) or binding protein SEQ ID NO: 28 (212896) showed homogenous elution profile with a singular peak. The peak maximum was at pH 3.4 in a pH gradient profile. 96% of captured RBD eluted at pH 3.4. For 213147 (SEQ ID NO: 32; comprises the dimer of 212728/SEQ ID NO: 18), the peak maximum was at about pH 4.3 in a pH gradient profile. The eluted RDB-His target showed high purity. For 213152 (SEQ ID NO: 33; comprises the dimer of 213103/SEQ ID NO: 9), the peak maximum was at about pH 3.9 in a pH gradient profile. The eluted RDB-His target showed high purity.

DBC 10%/Elution. The resin with immobilized fusion protein 212896 comprising binding protein SEQ ID NO: 28 was equilibrated in 1×PBS, pH 7.3. Expressed SARS-Cov2-RBD-His in cell culture supernatant was applied on column until 100% target breakthrough. The bound protein was eluted at 100 mM citric acid buffer pH 3.4 in a single step. The static binding capacity (SBC) was determined by the mass eluted protein calculate by UV280 nm absorption and the extinction coefficient of the target in 20 mM Na₂HPO, 150 mM NaCl, pH 6.5 or 1×PBS, pH 7.3 at 10% breakthrough at 6 min residence time. The S1 domain was eluted with 100 mM acetic acid, 150 mM NaCl, 10% (v/v) propylene glycole, pH 4.0, followed by 100 mM citrate acid, pH 2.0 or with

100 mM acetic acid pH 4.0, followed by 100 mM citrate acid, pH 2.0. The static binding capacity was about 10.7 mg/ml.

In a similar experiment, CoV-2 protein RBD was injected onto coupled resin with fusion proteins 217800, 217799, 217798, and the trimer 217797 of SEQ ID NO: 7 until 10% target breakthrough at 6 min residence time. Loaded SARS CoV-2 protein RBD was quantified and calculated as dynamic binding capacity DBC10%. The DBC10% at 6 min residence time with 1 mg/ml SARS CoV-2 protein RBD was 15.1 mg/ml for 217800, 17 mg/ml for 217799, 10.5 mg/ml for 217798, and 25.8 mg/ml for the trimer of SEQ ID NO: 7.

Caustic stability. The fusion protein 212896 comprising binding protein SEQ ID NO: 28 was coupled to Praesto™ Epoxy 85 as described above and treated with 0.1 M NaOH for at least 10 h at RT. The remaining RBD binding capacity was 87% (equals 40 CIP cycles).

Praesto 85 epoxy resin with immobilized 23.1 mg/ml 217800 was incubated with 0.1 M NaOH for 10 h at room temperature (22° C.+/−3° C.). After 10 h in strong alkaline solution, 217800 showed 22% reduction in binding capacity for RBD (remaining RBD binding capacity 78%). 15.1 mg/ml of immobilized 217799 showed a reduction of 28% in binding capacity for RBD after the incubation (remaining binding capacity 72%). 18.6 mg/ml of immobilized trimer 217797 of SEQ ID NO: 7 showed a reduction of 26% in binding capacity for RBD after the (remaining binding capacity 74%).

Purity of eluted fraction. 10 μg of neutralized eluted fraction from Praesto85_212896 (comprising monomer of SEQ ID NO: 28), SARS-CoV2-RBD was used as sample. Column: Superdex 200 increased, Tricorn 5/150. Running buffer: 1×PBS, pH 7.3. Results: The purity of the eluted fraction from Praesto-Expoxy 85_212896 was as high as at least 94% after a single chromatography step. The eluted fraction shows high homogeneity after the first purification step from cell culture supernatant (size exclusion chromatography).

Target characterization after elution (RBD) by SDS-PAGE. 2, 1, and 0.5 μg of neutralized eluted fraction from Praesto 85_212896, SARS CoV2 was used as sample. Gel-System: NuPAGE system (Invitrogen), 4-12% Bis-Tris-Gel. Staining: Coomassie Blue R250. Densitometric evaluation software: TotalLab 1D. No impurities after AIC were detectable in SDS-PAGE.

Target characterization after elution (RBD) by binding analysis (SPR). Sample: 100 mM of neutralized eluted fraction from Praesto 85_212896 (comprising monomer of SEQ ID NO: 28), SARS CoV2 was used as sample and analyzed for ACE2 binding. Senor surface: immobilized Protein A (CM5 Chip). Target: human ACE2-Fc (Aero Biosystems, Cat. No.: AC-H5257). Results: The eluted fraction of RBD-His showed binding to human ACE2 receptor. The qualitative binding was confirmed.

Further, sample neutralized eluted fraction from Praesto 85_212896, SARS CoV2 was analyzed for ACE2 binding, after capturing from cell culture supernatant and acid elution at pH 3.4. Senor surface: immobilized Protein A (SPR, CM5 Chip). Target: human ACE2-Fc (Aero Biosystems, Cat. No.: AC-H5257), purified SARS-Cov2-RBD-His was as standard reference. Results: The eluted fraction of RBD-His showed comparable binding to human ACE2 receptor and to purified target. The purification of SARS Cov2-RBD via Praesto_212896 has no negative influence on target binding activity.

### Example 5. Ligand Detection in Protein A ELISA (Leaching Assay)

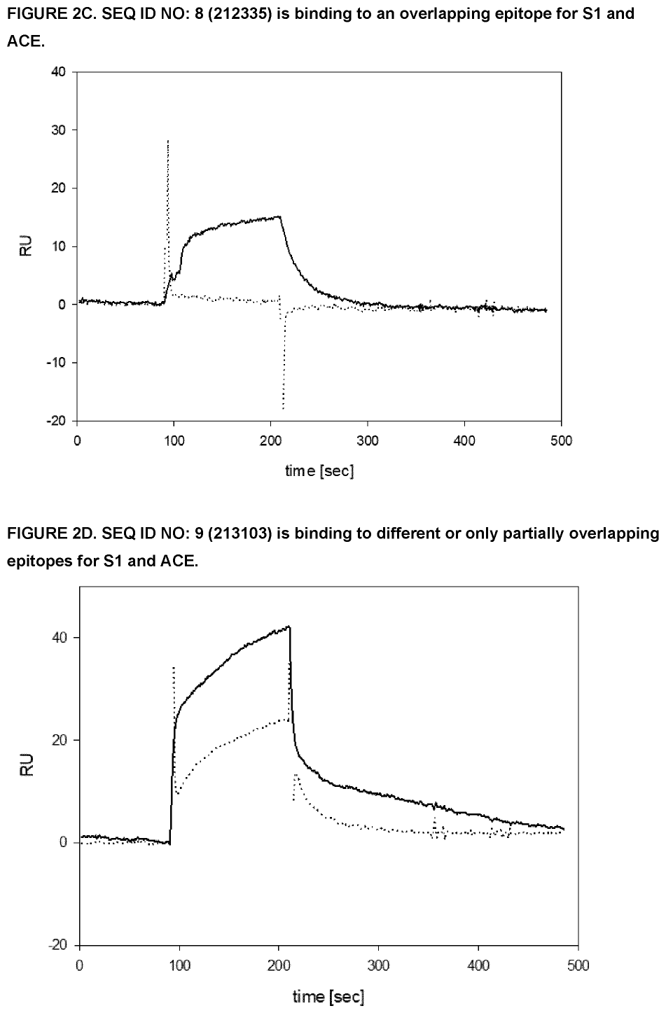

To determine low levels of leached variants in affinity chromatography is important for obtaining reliable results. Protein A ELISA Kits for the detection of native and recombinant Protein A (Repligen, Cat. No. 9000-1) were used for leaching assays according to manufacturer's instructions, except using 0.1% PBST as dilution buffer. Samples: 212896 (comprising monomer of SEQ ID NO: 28), 213147 (comprising monomers of SEQ ID NO: 26 and SEQ ID NO: 3), 213152 (comprising monomers of SEQ NO: 21 and SEQ ID NO: 22), concentration 1.6 ng/ml. All variants showed good detectability in PBST buffer, comparable to rProtein A standard. The detection signal of described variants is illustrated in FIG. 3.

### Example 6. Inhibition of ACE2 Binding

Spike protein binding proteins were immobilized on a CM-5 sensor chip (GE Healthcare) using NHS/EDC after PDEA activation as described above (Example 3). An equimolar mixture of 51-His and ACE2 or RBD-His and ACE2 was incubated for 1 h at RT. The mixture was applied to the chip and the binding of spike protein binding proteins analyzed. The binding signal is reduced upon binding to the same epitope. FIG. 2 shows the competitive binding study for 212434 (SEQ ID NO: 1), 212361 (SEQ ID NO: 7), 212335 (SEQ ID NO: 8), 213103 (SEQ ID NO: 9), and 212860 (SEQ ID NO: 17). The spike protein binding proteins 212434, 212361, 212860, and 212335 bind to the same or an overlapping epitope for 51 or RBD and ACE. Further, fusion protein of SEQ ID NO: 30 binds to the same or an overlapping epitope for 51 or RBD and ACE. The dimeric spike protein binding proteins 212361, 212860, 212335 have the same N-terminal monomer (SEQ ID NO: 2) having A11Y, E15K, P20W, Q49R, and D53T corresponding to SEQ ID NO: 15.

1. A binding protein for the spike protein of severe acute respiratory syndrome corona virus 2 (SARS-Cov-2) comprising an amino acid sequence with at least 92% sequence identity to any one of SEQ ID NOs: 2, 4, 5, 6, 23, and 40-42, wherein the binding protein has a binding affinity of less than 500 nM for the spike protein or domains thereof.

2. The binding protein for the spike protein according to claim 1, wherein the binding protein has a binding affinity of less than 500 nM for the S1 domain of the spike protein.

3. The binding protein for the spike protein according to claim 1, wherein 2, 3, 4, 5, or 6 binding proteins for the spike protein are linked to each other.

4. The binding protein for the spike protein according to claim 3, wherein the binding protein is a homo-multimer or a hetero-multimer.

5. The binding protein for the spike protein according to item 4, wherein the binding protein is a hetero-multimer that comprises an amino acid sequence with at least 92% sequence identity to an amino acid sequence selected from the group consisting of SEQ ID NOs: 2, 40, and 41 linked to an amino acid sequence with at least at least 92% sequence identity to an amino acid sequence selected from the group consisting of SEQ ID NOs: 4, 5, 6, 23, and 42.

6. The binding protein for the spike protein according to claim 5, wherein the binding protein comprises amino acid sequences with at least 80%, preferably at least 92% sequence identity to any one of SEQ ID NOs: 7, 8, 17, 43-47, 66, and 67.

7. The binding protein for the spike protein according to claim 1, wherein the binding protein is fused to or conjugated to at least one further molecule.

8. A method for diagnosing or treating a disease related to or caused by SARS CoV-2 or a variant thereof, the method comprising administering to a subject in need thereof, an effective amount of the binding protein for the spike protein according to claim 1.

9. (canceled)

10. An affinity separation matrix comprising a binding protein for the spike protein according to claim 1.

11. (canceled)

12. A method of affinity purification of spike protein or a domain thereof or of a particle comprising spike protein or a domain thereof, optionally a virus particle, the method comprising:
(a) providing a liquid that contains a spike protein or a domain thereof (e.g. a particle comprising spike protein or domain);
(b) providing an affinity separation matrix comprising at least one binding protein for spike protein or a domain thereof according to claim 1 coupled to said affinity separation matrix;
(c) contacting said affinity separation matrix with the liquid under conditions that permit binding of the at least one binding protein for spike protein or a domain thereof according to claim 1; and
(d) eluting said spike protein or a domain thereof or particle comprising the spike protein or domain thereof from said affinity purification matrix.

13. (canceled)

14. A method for analyzing the presence of spike protein or a domain thereof or particle comprising a spike protein or domain thereof in liquid samples, the method comprising the following steps:
providing a liquid that contains spike protein or a domain thereof or particle comprising a spike protein or domain,
(ii) providing the binding protein for the spike protein according to claim 1,
(iii) contacting the liquid of (i) with the binding protein for the spike protein or a domain thereof according to claim 1 under conditions that permit binding of the binding protein to the spike protein or a domain thereof,
(iv) isolating the complex of spike protein or a domain thereof or particle and the binding protein for the spike protein or a domain thereof according to claim 1, and
(v) determining the amount of the binding protein for the spike protein according to claim 1 in the liquid of (i).

15. A polynucleotide encoding the binding protein according to claim 1.

16. The binding protein for the spike protein according to claim 2, wherein the binding protein has a binding affinity of less than 100 nM for the S1 domain of the spike protein.

17. The binding protein for the spike protein according to claim 2, wherein the binding protein has a binding affinity of less than 500 nM for the for the receptor binding domain (RBD) of the spike protein.

18. The binding protein for the spike protein according to claim 17, wherein the binding protein has a binding affinity of less than 100 nM for the receptor binding domain (RBD) of the spike protein.

19. The binding protein for the spike protein according to item 5, wherein the binding protein is a hetero-dimer that comprises an amino acid sequence with at least 92% sequence identity to an amino acid sequence selected from the group consisting of SEQ ID NOs: 2, 40, and 41 linked to an amino acid sequence with at least at least 92% sequence identity to an amino acid sequence selected from the group consisting of SEQ ID NOs: 4, 5, 6, 23, and 42.

20. The binding protein for the spike protein according to claim 7, wherein the at least one further molecule is selected from the group consisting of:
(a) a non-Immunoglobulin (Ig)-binding protein;
(b) a diagnostically active moiety, optionally a radionuclide, fluorescent protein, photosensitizer, dye, enzyme, or any combination thereof;
(c) a therapeutically active moiety, optionally a monoclonal antibody or a fragment thereof, a binding protein, a receptor or receptor domain, a receptor binding ligand or antagonist, a radionuclide, a cytotoxic compound, a cytokine, a chemokine, an enzyme, or a derivative of any of the therapeutically active moieties thereof, or any combination thereof.


SEQUENCE LISTING (72 sequences)

SEQ ID NO: 1 - protein, 58 aa, Artificial Sequence
  Artificially synthesized spike protein binding protein 212434
NAAVLDIAQHSATEEIWWLPNLTKQQKVWFIMSLTQDPSVSKEVLGEAQKLNDSQAPK

SEQ ID NO: 2 - protein, 58 aa, Artificial Sequence
  Artificially synthesized spike protein binding protein
IAAKFDEAQQYAFYKILHLWNLTEEQRNAFIQSLRDDPSVSLEVLGEARKLNTSQAPK

SEQ ID NO: 3 - protein, 58 aa, Artificial Sequence
  Artificially synthesized spike protein binding protein
IAAKFDEAQLAAFQEIRHLPNLTEEQREAFITSLYDDPSVSLEVLGEAQKLNDSQAPK

SEQ ID NO: 4 - protein, 58 aa, Artificial Sequence
  Artificially synthesized spike protein binding protein
  misc_feature (17)..(17): Xaa can be any naturally occurring amino acid
  misc_feature (28)..(28): Xaa can be any naturally occurring amino acid
  misc_feature (32)..(32): Xaa can be any naturally occurring amino acid
  misc_feature (35)..(35): Xaa can be any naturally occurring amino acid
IAAKFDEAQYAAFLEIXHLPNLTEEQRXAFIXSLXDDPSVSLEVLGEAQKLNDSQAPK

SEQ ID NO: 5 - protein, 58 aa, Artificial Sequence
  Artificially synthesized spike protein binding protein
IAAKFDEAQYAAFLEIVHLPNLTEEQRQAFIASLWDDPSVSLEVLGEAQKLNDSQAPK

SEQ ID NO: 6 - protein, 58 aa, Artificial Sequence
  Artificially synthesized spike protein binding protein
IAAKFDEAQYAAFLEIHHLPNLTEEQRHAFILSLYDDPSVSLEVLGEAQKLNDSQAPK

SEQ ID NO: 7 - protein, 116 aa, Artificial Sequence
  Artificially synthesized spike protein binding protein 212361
IAAKFDEAQQYAFYKILHLWNLTEEQRNAFIQSLRDDPSVSLEVLGEARKLNTSQAPKIAAKFDEAQYAAFLEIVHLPNLTEEQRQAFIASLWDDPSVSLEVLGEAQKLNDSQAPK

SEQ ID NO: 8 - protein, 116 aa, Artificial Sequence
  Artificially synthesized spike protein binding protein 212335
IAAKFDEAQQYAFYKILHLWNLTEEQRNAFIQSLRDDPSVSLEVLGEARKLNTSQAPKIAAKFDEAQYAAFLEIHHLPNLTEEQRHAFILSLYDDPSVSLEVLGEAQKLNDSQAPK

SEQ ID NO: 9 - protein, 116 aa, Artificial Sequence
  Artificially synthesized spike protein binding protein 213103
IAAKFDEAQQWAFYEILHLENLTEEQRNAFIQSLRDDPSVSREVLGEATKLNTSQAPKIAAKFDEAQLAAFQEIRHLPNLTEEQREAFITSLYDDPSVSLEVLGEAQKLNDSQAPK

SEQ ID NO: 10 - protein, 1273 aa, Artificial Sequence
  Artificially synthesized spike glycoprotein S
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 11 - protein, 670 aa, Artificial Sequence
  Artificially synthesized S1 domain of spike protein
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 12 - protein, 223 aa, Artificial Sequence
  Artificially synthesized receptor binding domain (RBD) of spike protein
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 13 - protein, 58 aa, Artificial Sequence
  Artificially synthesized PAdelFc
IAAQHDKDQSAADSEILHLPNLTEEQRNAFRQSLSDDPSVSLEILAEAKKLNDAQAPK

SEQ ID NO: 14 - protein, 58 aa, Artificial Sequence
  Artificially synthesized PAdelFc non-immunoglobulin binding protein
IAAQHDKIQQAADKEILHLPNLTEEQRNKFRQSLRDDPSVSAEILAEAKKLNDAQAPK

SEQ ID NO: 15 - protein, 58 aa, Artificial Sequence
  Artificially synthesized Ig binding protein cs27 as scaffold
IAAKFDEAQQAAFYEILHLPNLTEEQRNAFIQSLRDDPSVSLEVLGEAQKLNDSQAPK

SEQ ID NO: 16 - protein, 58 aa, Artificial Sequence
  Artificially synthesized Ig binding protein C27 as scaffold
NAAKFDEAQQSAFYEILHLPNLTEEQRNAFIQSLKDDPSVSKEVLGEAQKLNDSQAPK

SEQ ID NO: 17 - protein, 116 aa, Artificial Sequence
  Artificially synthesized spike protein binding protein 212860
IAAKFDEAQQYAFYKILHLWNLTEEQRNAFIQSLRDDPSVSLEVLGEARKLNTSQAPKIAAKFDEAQYAAFLEIQHLPNLTEEQRQAFIVSLADDPSVSLEVLGEAQKLNDSQAPK

SEQ ID NO: 18 - protein, 116 aa, Artificial Sequence
  Artificially synthesized spike protein binding protein 212728
IAAKFDEAQLAAFEEIRHLPNLTEEQREAFITSLYDDPSVSLEVLGEAQKLNDSQAPKIAAKFDEAQQAAFYEILHLPNPTEWQRNYFIQQLRDDPSVSLAVLGRAQVLNDSQAPK

SEQ ID NO: 19 - protein, 116 aa, Artificial Sequence
  Artificially synthesized spike protein binding protein 212522
IAAKFDEAQQAAFYEILHLPNLTELQRNWFIQLLRDDPSVSLQVLGHAQYLNDSQAPKIAAKFDEAQQYAFYQILHLHNLTEEQRNAFIQSLRDDPSVSHEVLGEAKKLNESQAPK

SEQ ID NO: 20 - protein, 58 aa, Artificial Sequence
  Artificially synthesized SPike protein binding protein
  Xaa (14)..(14)
  Xaa (14)..(14): Xaa may be any amino acid, in some embodiments Q or E
IAAKFDEAQLAAFXEIRHLPNLTEEQREAFITSLYDDPSVSLEVLGEAQKLNDSQAPK

SEQ ID NO: 21 - protein, 58 aa, Artificial Sequence
  Artificially synthesized Spike protein binding protein
IAAKFDEAQLAAFEEIRHLPNLTEEQREAFITSLYDDPSVSLEVLGEAQKLNDSQAPK

SEQ ID NO: 22 - protein, 58 aa, Artificial Sequence
  Artificially synthesized Spike protein binding protein
IAAKFDEAQQAAFYEILHLPNPTEWQRNYFIQQLRDDPSVSLAVLGRAQVLNDSQAPK

SEQ ID NO: 23 - protein, 58 aa, Artificial Sequence
  Artificially synthesized Spike protein binding protein
IAAKFDEAQYAAFLEIQHLPNLTEEQRQAFIVSLADDPSVSLEVLGEAQKLNDSQAPK

SEQ ID NO: 24 - protein, 58 aa, Artificial Sequence
  Artificially synthesized Spike protein binding protein
IAAKFDEAQQAAFYEILHLPNLTELQRNWFIQLLRDDPSVSLQVLGHAQYLNDSQAPK

SEQ ID NO: 25 - protein, 58 aa, Artificial Sequence
  Artificially synthesized Spike protein binding protein
IAAKFDEAQQYAFYQILHLHNLTEEQRNAFIQSLRDDPSVSHEVLGEAKKLNESQAPK

SEQ ID NO: 26 - protein, 58 aa, Artificial Sequence
  Artificially synthesized Spike protein binding protein
IAAKFDEAQQWAFYEILHLENLTEEQRNAFIQSLRDDPSVSREVLGEATKLNTSQAPK

SEQ ID NO: 27 - protein, 56 aa, Artificial Sequence
  Artificially synthesized Spike binding protein 212434 Ndel2
AVLDIAQHSATEEIWWLPNLTKQQKVWFIMSLTQDPSVSKEVLGEAQKLNDSQAPK

SEQ ID NO: 28 - protein, 56 aa, Artificial Sequence
  Artificially synthesized spike binding protein 212896
AVLDIDQHAATEEIWWLPNLTKQQKVWFIMSLTQDPSVSLEILAEAKKLNDAQAPK

SEQ ID NO: 29 - protein, 231 aa, Artificial Sequence
  Artificially synthesized fusion protein 212895 (fusion of 212434 with (PAdelFc)3)
AVLDIAQHSATEEIWWLPNLTKQQKVWFIMSLTQDPSVSKEVLGEAQKLNDSQAPKIAAQHDKIQQAADKEILHLPNLTEEQRNKFRQSLRDDPSVSAEILAEAKKLNDAQAPKIAAQHDKIQQAADKEILHLPNLTEEQRNKFRQSLRDDPSVSAEILAEAKKLNDAQAPKIAAQHDKIQQAADKEILHLPNLTEEQRNKFRQSLRDDPSVSAEILAEAKKLNDAQAPKC

SEQ ID NO: 30 - protein, 231 aa, Artificial Sequence
  Artificially synthesized fusion protein 212896 (fusion of 212896 with (PAdelFc)3)
AVLDIDQHAATEEIWWLPNLTKQQKVWFIMSLTQDPSVSLEILAEAKKLNDAQAPKIAAQHDKIQQAADKEILHLPNLTEEQRNKFRQSLRDDPSVSAEILAEAKKLNDAQAPKIAAQHDKIQQAADKEILHLPNLTEEQRNKFRQSLRDDPSVSAEILAEAKKLNDAQAPKIAAQHDKIQQAADKEILHLPNLTEEQRNKFRQSLRDDPSVSAEILAEAKKLNDAQAPKC

SEQ ID NO: 31 - protein, 365 aa, Artificial Sequence
  Artificially synthesized fusion protein 212897 (fusion of 212434 dimer with (PAdelFc)4)
AVLDIAQHSATEEIWWLPNLTKQQKVWFIMSLTQDPSVSKEVLGEAQKLNDSQAPKQAPKVDAKFDQAPKVDAKFDAVLDIAQHSATEEIWWLPNLTKQQKVWFIMSLTQDPSVSKEVLGEAQKLNDSQAPKIAAQHDKIQQAADKEILHLPNLTEEQRNKFRQSLRDDPSVSAEILAEAKKLNDAQAPKIAAQHDKIQQAADKEILHLPNLTEEQRNKFRQSLRDDPSVSAEILAEAKKLNDAQAPKIAAQHDKIQQAADKEILHLPNLTEEQRNKFRQSLRDDPSVSAEILAEAKKLNDAQAPKIAAQHDKIQQAADKEILHLPNLTEEQRNKFRQSLRDDPSVSAEILAEAKKLNDAQAPKC

SEQ ID NO: 32 - protein, 230 aa, Artificial Sequence
  Artificially synthesized fusion protein 213147 (213103 with (PAdelFc)2)
AKFDEAQQWAFYEILHLENLTEEQRNAFIQSLRDDPSVSREVLGEATKLNTSQAPKIAAKFDEAQLAAFQEIRHLPNLTEEQREAFITSLYDDPSVSLEVLGEAQKLNDSQAPKIAAQHDKIQQAADKEILHLPNLTEEQRNKFRQSLRDDPSVSAEILAEAKKLNDAQAPKIAAQHDKIQQAADKEILHLPNLTEEQRNKFRQSLRDDPSVSAEILAEAKKLNDAQAPK

SEQ ID NO: 33 - protein, 230 aa, Artificial Sequence
  Artificially synthesized fusion protein 213152 (212728 with (PAdelFc)2)
AKFDEAQLAAFEEIRHLPNLTEEQREAFITSLYDDPSVSLEVLGEAQKLNDSQAPKIAAKFDEAQQAAFYEILHLPNPTEWQRNYFIQQLRDDPSVSLAVLGRAQVLNDSQAPKIAAQHDKIQQAADKEILHLPNLTEEQRNKFRQSLRDDPSVSAEILAEAKKLNDAQAPKIAAQHDKIQQAADKEILHLPNLTEEQRNKFRQSLRDDPSVSAEILAEAKKLNDAQAPK

SEQ ID NO: 34 - protein, 114 aa, Artificial Sequence
  Artificially synthesized spike binding protein 213103 Ndel2
AKFDEAQQWAFYEILHLENLTEEQRNAFIQSLRDDPSVSREVLGEATKLNTSQAPKIAAKFDEAQLAAFQEIRHLPNLTEEQREAFITSLYDDPSVSLEVLGEAQKLNDSQAPK

SEQ ID NO: 35 - protein, 114 aa, Artificial Sequence
  Artificially synthesized spike binding protein 212728 Ndel2
AKFDEAQLAAFEEIRHLPNLTEEQREAFITSLYDDPSVSLEVLGEAQKLNDSQAPKIAAKFDEAQQAAFYEILHLPNPTEWQRNYFIQQLRDDPSVSLAVLGRAQVLNDSQAPK

SEQ ID NO: 36 - protein, 116 aa, Artificial Sequence
  Artificially synthesized spike binding protein 212880 (SEQ37+SEQ3)
IAAKFDEAQQLAFYLILHLINLTEEQRNAFIQSLRDDPSVSLEVLGEAYKLNLSQAPKIAAKFDEAQLAAFQEIRHLPNLTEEQREAFITSLYDDPSVSLEVLGEAQKLNDSQAPK

SEQ ID NO: 37 - protein, 58 aa, Artificial Sequence
  Artificially synthesized spike binding protein 212880 monomer
IAAKFDEAQQLAFYLILHLINLTEEQRNAFIQSLRDDPSVSLEVLGEAYKLNLSQAPK

SEQ ID NO: 38 - protein, 172 aa, Artificial Sequence
  Artificially synthesized fusion protein of SEQ28 with (PAdelFc)2
AVLDIDQHAATEEIWWLPNLTKQQKVWFIMSLTQDPSVSLEILAEAKKLNDAQAPKIAAQHDKIQQAADKEILHLPNLTEEQRNKFRQSLRDDPSVSAEILAEAKKLNDAQAPKIAAQHDKIQQAADKEILHLPNLTEEQRNKFRQSLRDDPSVSAEILAEAKKLNDAQAPK

SEQ ID NO: 39 - protein, 230 aa, Artificial Sequence
  Artificially synthesized fusion protein of SEQ28-PAdelFc-SEQ28-PAdelFc
AVLDIDQHAATEEIWWLPNLTKQQKVWFIMSLTQDPSVSLEILAEAKKLNDAQAPKIAAQHDKIQQAADKEILHLPNLTEEQRNKFRQSLRDDPSVSAEILAEAKKLNDAQAPKIAAVLDIDQHAATEEIWWLPNLTKQQKVWFIMSLTQDPSVSLEILAEAKKLNDAQAPKIAAQHDKIQQAADKEILHLPNLTEEQRNKFRQSLRDDPSVSAEILAEAKKLNDAQAPK

SEQ ID NO: 40 - protein, 56 aa, Artificial Sequence
  Artificially synthesized spike protein binding protein
AKFDEAQQYAFYKILHLWNLTEEQRNAFIQSLRDDPSVSLEVLGEARKLNTSQAPK

SEQ ID NO: 41 - protein, 56 aa, Artificial Sequence
  Artificially synthesized spike binding protein 217800-1
AKFDEAQQYAFYKILHLWNLTEEQRNAFIQSLRDDPSVSLEILAEARKLNTAQAPK

SEQ ID NO: 42 - protein, 58 aa, Artificial Sequence
  Artificially synthesized spike protein binding protein 217800-2
IAAKFDEAQYAAFLEIVHLPNLTEEQRQAFIASLWDDPSVSLEILAEAKKLNDAQAPK

SEQ ID NO: 43 - protein, 114 aa, Artificial Sequence
  Artificially synthesized spike protein binding protein 217800
AKFDEAQQYAFYKILHLWNLTEEQRNAFIQSLRDDPSVSLEILAEARKLNTAQAPKIAAKFDEAQYAAFLEIVHLPNLTEEQRQAFIASLWDDPSVSLEILAEAKKLNDAQAPK

SEQ ID NO: 44 - protein, 114 aa, Artificial Sequence
  Artificially synthesized spike protein binding protein 217798
AKFDEAQQYAFYKILHLWNLTEEQRNAFIQSLRDDPSVSLEVLGEARKLNTSQAPKIAAKFDEAQYAAFLEIVHLPNLTEEQRQAFIASLWDDPSVSLEILAEAKKLNDAQAPK

SEQ ID NO: 45 - protein, 114 aa, Artificial Sequence
  Artificially synthesized spike protein binding protein (SEQ2 plus SEQ3)
AKFDEAQQYAFYKILHLWNLTEEQRNAFIQSLRDDPSVSLEVLGEARKLNTSQAPKIAAKFDEAQLAAFQEIRHLPNLTEEQREAFITSLYDDPSVSLEVLGEAQKLNDSQAPK

SEQ ID NO: 46 - protein, 230 aa, Artificial Sequence
  Artificially synthesized spike protein binding protein (217800 tetramer)
AKFDEAQQYAFYKILHLWNLTEEQRNAFIQSLRDDPSVSLEILAEARKLNTAQAPKIAAKFDEAQYAAFLEIVHLPNLTEEQRQAFIASLWDDPSVSLEILAEAKKLNDAQAPKIAAKFDEAQQYAFYKILHLWNLTEEQRNAFIQSLRDDPSVSLEILAEARKLNTAQAPKIAAKFDEAQYAAFLEIVHLPNLTEEQRQAFIASLWDDPSVSLEILAEAKKLNDAQAPK

SEQ ID NO: 47 - protein, 347 aa, Artificial Sequence
  Artificially synthesized 212361 trimer (217797)
AKFDEAQQYAFYKILHLWNLTEEQRNAFIQSLRDDPSVSLEVLGEARKLNTSQAPKIAAKFDEAQYAAFLEIVHLPNLTEEQRQAFIASLWDDPSVSLEVLGEAQKLNDSQAPKIAAKFDEAQQYAFYKILHLWNLTEEQRNAFIQSLRDDPSVSLEVLGEARKLNTSQAPKIAAKFDEAQYAAFLEIVHLPNLTEEQRQAFIASLWDDPSVSLEVLGEAQKLNDSQAPKIAAKFDEAQQYAFYKILHLWNLTEEQRNAFIQSLRDDPSVSLEVLGEARKLNTSQAPKIAAKFDEAQYAAFLEIVHLPNLTEEQRQAFIASLWDDPSVSLEVLGEAQKLNDSQAPKC

SEQ ID NO: 48 - protein, 231 aa, Artificial Sequence
  Artificially synthesized Fusion protein 217238 (212361 fused to (PAdelFc)2)
AKFDEAQQYAFYKILHLWNLTEEQRNAFIQSLRDDPSVSLEVLGEARKLNTSQAPKIAAKFDEAQYAAFLEIVHLPNLTEEQRQAFIASLWDDPSVSLEVLGEAQKLNDSQAPKIAAQHDKIQQAADKEILHLPNLTEEQRNKFRQSLRDDPSVSAEILAEAKKLNDAQAPKIAAQHDKIQQAADKEILHLPNLTEEQRNKFRQSLRDDPSVSAEILAEAKKLNDAQAPKC

SEQ ID NO: 49 - protein, 231 aa, Artificial Sequence
  Artificially synthesized Fusion protein 218898
AKFDEAQQYAFYKILHLWNLTEEQRNAFIQSLRDDPSVSLEVLGEARKLNTSQAPKIAAKFDEAQYAAFLEIVHLPNLTEEQRQAFIASLWDDPSVSLEILAEAKKLNDAQAPKIAAQHDKIQQAADKEILHLPNLTEEQRNKFRQSLRDDPSVSAEILAEAKKLNDAQAPKIAAQHDKIQQAADKEILHLPNLTEEQRNKFRQSLRDDPSVSAEILAEAKKLNDAQAPKC

SEQ ID NO: 50 - protein, 347 aa, Artificial Sequence
  Artificially synthesized Fusion protein 217799
AKFDEAQQYAFYKILHLWNLTEEQRNAFIQSLRDDPSVSLEVLGEARKLNTSQAPKIAAKFDEAQYAAFLEIVHLPNLTEEQRQAFIASLWDDPSVSLEILAEAKKLNDAQAPKIAAKFDEAQQYAFYKILHLWNLTEEQRNAFIQSLRDDPSVSLEVLGEARKLNTSQAPKIAAKFDEAQYAAFLEIVHLPNLTEEQRQAFIASLWDDPSVSLEILAEAKKLNDAQAPKIAAQHDKIQQAADKEILHLPNLTEEQRNKFRQSLRDDPSVSAEILAEAKKLNDAQAPKIAAQHDKIQQAADKEILHLPNLTEEQRNKFRQSLRDDPSVSAEILAEAKKLNDAQAPKC

SEQ ID NO: 51 - protein, 231 aa, Artificial Sequence
  Artificially synthesized Fusion protein 217800
AKFDEAQQYAFYKILHLWNLTEEQRNAFIQSLRDDPSVSLEILAEARKLNTAQAPKIAAKFDEAQYAAFLEIVHLPNLTEEQRQAFIASLWDDPSVSLEILAEAKKLNDAQAPKIAAQHDKIQQAADKEILHLPNLTEEQRNKFRQSLRDDPSVSAEILAEAKKLNDAQAPKIAAQHDKIQQAADKEILHLPNLTEEQRNKFRQSLRDDPSVSAEILAEAKKLNDAQAPKC

SEQ ID NO: 52 - protein, 231 aa, Artificial Sequence
  Artificially synthesized Fusion protein SEQ28xSEQ43xPAdelFc
AVLDIDQHAATEEIWWLPNLTKQQKVWFIMSLTQDPSVSLEILAEAKKLNDAQAPKIAAKFDEAQQYAFYKILHLWNLTEEQRNAFIQSLRDDPSVSLEILAEARKLNTAQAPKIAAKFDEAQYAAFLEIVHLPNLTEEQRQAFIASLWDDPSVSLEILAEAKKLNDAQAPKIAAQHDKIQQAADKEILHLPNLTEEQRNKFRQSLRDDPSVSAEILAEAKKLNDAQAPKC

SEQ ID NO: 53 - protein, 231 aa, Artificial Sequence
  Artificially synthesized Fusion protein (SEQ43xSEQ28xPAdelFc)
AKFDEAQQYAFYKILHLWNLTEEQRNAFIQSLRDDPSVSLEILAEARKLNTAQAPKIAAKFDEAQYAAFLEIVHLPNLTEEQRQAFIASLWDDPSVSLEILAEAKKLNDAQAPKIAAVLDIDQHAATEEIWWLPNLTKQQKVWFIMSLTQDPSVSLEILAEAKKLNDAQAPKIAAQHDKIQQAADKEILHLPNLTEEQRNKFRQSLRDDPSVSAEILAEAKKLNDAQAPKC

SEQ ID NO: 54 - protein, 288 aa, Artificial Sequence
  Artificially synthesized Fusion protein (217800 dimer x PAdelFc)
AKFDEAQQYAFYKILHLWNLTEEQRNAFIQSLRDDPSVSLEILAEARKLNTAQAPKIAAKFDEAQYAAFLEIVHLPNLTEEQRQAFIASLWDDPSVSLEILAEAKKLNDAQAPKIAAKFDEAQQYAFYKILHLWNLTEEQRNAFIQSLRDDPSVSLEILAEARKLNTAQAPKIAAKFDEAQYAAFLEIVHLPNLTEEQRQAFIASLWDDPSVSLEILAEAKKLNDAQAPKIAAQHDKIQQAADKEILHLPNLTEEQRNKFRQSLRDDPSVSAEILAEAKKLNDAQAPK

SEQ ID NO: 55 - protein, 346 aa, Artificial Sequence
  Artificially synthesized Fusion protein 217800 x PAdelFc
AKFDEAQQYAFYKILHLWNLTEEQRNAFIQSLRDDPSVSLEILAEARKLNTAQAPKIAAKFDEAQYAAFLEIVHLPNLTEEQRQAFIASLWDDPSVSLEILAEAKKLNDAQAPKIAAKFDEAQQYAFYKILHLWNLTEEQRNAFIQSLRDDPSVSLEILAEARKLNTAQAPKIAAKFDEAQYAAFLEIVHLPNLTEEQRQAFIASLWDDPSVSLEILAEAKKLNDAQAPKIAAQHDKIQQAADKEILHLPNLTEEQRNKFRQSLRDDPSVSAEILAEAKKLNDAQAPKIAAQHDKIQQAADKEILHLPNLTEEQRNKFRQSLRDDPSVSAEILAEAKKLNDAQAPK

SEQ ID NO: 56 - protein, 229 aa, Artificial Sequence
  Artificially synthesized Fusion protein 217382 (212361 W20P) (no binding to spike protein)
AKFDEAQQYAFYKILHLPNLTEEQRNAFIQSLRDDPSVSLEVLGEARKLNTSQAPKIAAKFDEAQYAAFLEIVHLPNLTEEQRQAFIASLWDDPSVSLEVLGEAQKLNDSQAPKAQHDKIQQAADKEILHLPNLTEEQRNKFRQSLRDDPSVSAEILAEAKKLNDAQAPKIAAQHDKIQQAADKEILHLPNLTEEQRNKFRQSLRDDPSVSAEILAEAKKLNDAQAPKC

SEQ ID NO: 57 - protein, 114 aa, Artificial Sequence
  Artificially synthesized Spike binding protein 212864 (LQRETY-group)
AKFDEAQQQAFYRILHLINLTEEQRNAFIQSLRDDPSVSTEVLGEAQKLNTSQAPKIAAKFDEAQLAAFQEIRHLPNLTEEQREAFITSLYDDPSVSLEVLGEAQKLNDSQAPK

SEQ ID NO: 58 - protein, 114 aa, Artificial Sequence
  Artificially synthesized Spike protein binding protein 213102 (LQRETY-group)
AKFDEAQQAAFYLILHLHNLTEEQRNAFIQSLRDDPSVSYEVLGEAQKLNLSQAPKIAAKFDEAQLAAFQEIRHLPNLTEEQREAFITSLYDDPSVSLEVLGEAQKLNDSQAPK

SEQ ID NO: 59 - protein, 114 aa, Artificial Sequence
  Artificially synthesized spike protein binding protein 213101 (LQRETY-group)
AKFDEAQQQAFYHILHLWNLTEEQRNAFIQSLRDDPSVSTEVLGEAEKLNISQAPKIAAKFDEAQLAAFQEIRHLPNLTEEQREAFITSLYDDPSVSLEVLGEAQKLNDSQAPK

SEQ ID NO: 60 - protein, 114 aa, Artificial Sequence
  Artificially synthesized spike protein binding protein 212853 (LQRETY-group)
AKFDEAQQYAFYWILHLWNLTEEQRNAFIQSLRDDPSVSWEVLGEAYKLNLSQAPKIAAKFDEAQLAAFQEIRHLPNLTEEQREAFITSLYDDPSVSLEVLGEAQKLNDSQAPK

SEQ ID NO: 61 - protein, 114 aa, Artificial Sequence
  Artificially synthesized spike protein binding protein 213100 (LQRETY group)
AKFDEAQLAAFQEIRHLPNLTEEQREAFITSLYDDPSVSLEVLGEAQKLNDSQAPKIAAKFDEAQQAAFYEILHLPNLTEKQRNHFIQILRDDPSVSLRVLGYAQHLNDSQAPK

SEQ ID NO: 62 - protein, 114 aa, Artificial Sequence
  Artificially synthesized spike protein binding protein 213099 (LQRETY-group)
AKFDEAQLAAFQEIRHLPNLTEEQREAFITSLYDDPSVSLEVLGEAQKLNDSQAPKIAAKFDEAQQAAFYEILHLPNLTEHQRNAFIQWLRDDPSVSLQVLGHAQQLNDSQAPK

SEQ ID NO: 63 - protein, 114 aa, Artificial Sequence
  Artificially synthesized Spike protein binding protein SEQ28 dimer
AVLDIDQHAATEEIWWLPNLTKQQKVWFIMSLTQDPSVSLEILAEAKKLNDAQAPKIAAVLDIDQHAATEEIWWLPNLTKQQKVWFIMSLTQDPSVSLEILAEAKKLNDAQAPK

SEQ ID NO: 64 - protein, 114 aa, Artificial Sequence
  Artificially synthesized spike protein binding protein SEQ ID NO 1dimer
AVLDIAQHSATEEIWWLPNLTKQQKVWFIMSLTQDPSVSKEVLGEAQKLNDSQAPKNAAVLDIAQHSATEEIWWLPNLTKQQKVWFIMSLTQDPSVSKEVLGEAQKLNDSQAPK

SEQ ID NO: 65 - protein, 58 aa, Artificial Sequence
  Artificially synthesized spike protein binding protein 434 consensus
  Xaa (8)..(8): Xaa may be selected from A or D
  Xaa (11)..(11): Xaa may be selected from S or A
  Xaa (42)..(42): Xaa may be selected from K or L
  Xaa (44)..(44): Xaa may be selected from V or I
  Xaa (46)..(46): Xaa may be selected from G or A
  Xaa (49)..(49): Xaa may be selected from Q or K
  Xaa (54)..(54): Xaa may be selected from S or A
NAAVLDIXQHXATEEIWWLPNLTKQQKVWFIMSLTQDPSVSXEXLXEAXKLNDXQAPK

SEQ ID NO: 66 - protein, 114 aa, Artificial Sequence
  Artificially synthesized Spike protein binding protein dimer 212361del2N
AKFDEAQQYAFYKILHLWNLTEEQRNAFIQSLRDDPSVSLEVLGEARKLNTSQAPKIAAKFDEAQYAAFLEIVHLPNLTEEQRQAFIASLWDDPSVSLEVLGEAQKLNDSQAPK

SEQ ID NO: 67 - protein, 114 aa, Artificial Sequence
  Artificially synthesized spike protein binding protein dimer 212335del2N
AKFDEAQQYAFYKILHLWNLTEEQRNAFIQSLRDDPSVSLEVLGEARKLNTSQAPKIAAKFDEAQYAAFLEIHHLPNLTEEQRHAFILSLYDDPSVSLEVLGEAQKLNDSQAPK

SEQ ID NO: 68 - protein, 347 aa, Artificial Sequence
  Artificially synthesized fusion protein of SEQ43 x (PAdelFc)3 x SEQ28
AKFDEAQQYAFYKILHLWNLTEEQRNAFIQSLRDDPSVSLEILAEARKLNTAQAPKIAAKFDEAQYAAFLEIVHLPNLTEEQRQAFIASLWDDPSVSLEILAEAKKLNDAQAPKIAAQHDKIQQAADKEILHLPNLTEEQRNKFRQSLRDDPSVSAEILAEAKKLNDAQAPKIAAQHDKIQQAADKEILHLPNLTEEQRNKFRQSLRDDPSVSAEILAEAKKLNDAQAPKIAAQHDKIQQAADKEILHLPNLTEEQRNKFRQSLRDDPSVSAEILAEAKKLNDAQAPKIAAVLDIDQHAATEEIWWLPNLTKQQKVWFIMSLTQDPSVSLEILAEAKKLNDAQAPKC

SEQ ID NO: 69 - protein, 289 aa, Artificial Sequence
  Artificially synthesized fusion protein of SEQ43 x (PAdelFc)2 x SEQ28
AKFDEAQQYAFYKILHLWNLTEEQRNAFIQSLRDDPSVSLEILAEARKLNTAQAPKIAAKFDEAQYAAFLEIVHLPNLTEEQRQAFIASLWDDPSVSLEILAEAKKLNDAQAPKIAAQHDKIQQAADKEILHLPNLTEEQRNKFRQSLRDDPSVSAEILAEAKKLNDAQAPKIAAQHDKIQQAADKEILHLPNLTEEQRNKFRQSLRDDPSVSAEILAEAKKLNDAQAPKIAAVLDIDQHAATEEIWWLPNLTKQQKVWFIMSLTQDPSVSLEILAEAKKLNDAQAPKC

SEQ ID NO: 70 - protein, 231 aa, Artificial Sequence
  Artificially synthesized fusion protein of SEQ43 x (PAdelFc)1 x SEQ28
AKFDEAQQYAFYKILHLWNLTEEQRNAFIQSLRDDPSVSLEILAEARKLNTAQAPKIAAKFDEAQYAAFLEIVHLPNLTEEQRQAFIASLWDDPSVSLEILAEAKKLNDAQAPKIAAQHDKIQQAADKEILHLPNLTEEQRNKFRQSLRDDPSVSAEILAEAKKLNDAQAPKIAAVLDIDQHAATEEIWWLPNLTKQQKVWFIMSLTQDPSVSLEILAEAKKLNDAQAPKC

SEQ ID NO: 71 - protein, 289 aa, Artificial Sequence
  Artificially synthesized fusion protein of SEQ28 x SEQ43 x (PAdelFc)2
AVLDIDQHAATEEIWWLPNLTKQQKVWFIMSLTQDPSVSLEILAEAKKLNDAQAPKIAAKFDEAQQYAFYKILHLWNLTEEQRNAFIQSLRDDPSVSLEILAEARKLNTAQAPKIAAKFDEAQYAAFLEIVHLPNLTEEQRQAFIASLWDDPSVSLEILAEAKKLNDAQAPKIAAQHDKIQQAADKEILHLPNLTEEQRNKFRQSLRDDPSVSAEILAEAKKLNDAQAPKIAAQHDKIQQAADKEILHLPNLTEEQRNKFRQSLRDDPSVSAEILAEAKKLNDAQAPKC

SEQ ID NO: 72 - protein, 289 aa, Artificial Sequence
  Artificially synthesized fusion protein of SEQ43 x SEQ28 x (PAdelFc)2
AKFDEAQQYAFYKILHLWNLTEEQRNAFIQSLRDDPSVSLEILAEARKLNTAQAPKIAAKFDEAQYAAFLEIVHLPNLTEEQRQAFIASLWDDPSVSLEILAEAKKLNDAQAPKIAAVLDIDQHAATEEIWWLPNLTKQQKVWFIMSLTQDPSVSLEILAEAKKLNDAQAPKIAAQHDKIQQAADKEILHLPNLTEEQRNKFRQSLRDDPSVSAEILAEAKKLNDAQAPKIAAQHDKIQQAADKEILHLPNLTEEQRNKFRQSLRDDPSVSAEILAEAKKLNDAQAPKC